(19) United States
(12) Patent Application Publication
(10) Pub. No.: US 2007/0198052 A1
(43) Pub. Date: Aug. 23, 2007

(54) Device and method for maintaining pressure onto a blood vessel

(76) Inventor: Shlomo Ben-David, Tel Aviv (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 10/598,999

(22) PCT Filed: Mar. 21, 2005
(86) PCT No.: PCT/IL05/00320

§ 371 Date: Mar. 29, 2007

(30) Foreign Application Priority Data
Mar. 22, 2004 (IL) 161010
Jun. 24, 2004 (IL) 162723
Jan. 3, 2005 (IL) 166106

(51) Int. Cl. A61B 17/03; A61B 17/08

(52) U.S. Cl. 606/201; 606/213

(57) ABSTRACT

A device and method for maintaining pressure onto a blood vessel is disclosed, and include a tissue-confining device having two parallel longitudinally extending bars which extend between a proximal end and a distal end and at least one strap attachable to tissue in the vicinity of the blood vessel and affixable to an element connected to the tissue-confining device, for retaining the tissue-confining device in compressing contact with tissue in the vicinity of the blood vessel. The device is suitable for inducing hemostasis and for maintaining post-hemostasis pressure, large saphenous vein closure, post-pseudoaneurysm closure, and conglutination of fragmented portions of a burst artery.

### FIELD OF THE INVENTION

The present invention relates to a device and method for maintaining pressure on a blood vessel, e.g. puncture site that has been sealed following a percutaneous medical procedure.

### BACKGROUND OF THE INVENTION

During several surgical procedures, for example in treatment of vascular diseases, it is a common practice to invade a blood vessel and introduce a treating or diagnostic device, e.g. balloons or various types of stents to operate on walls of the arteries, plaque removing devices, observation and flow diagnostic instruments, etc.

During such procedures, a blood vessel is punctured so as to allow introduction of an instrument therethrough, which is maneuvered thereinafter to the required site of operation. This is carried out in practice by introducing a guide sheath, through which the instrument can then be easily maneuvered to the site of interest.

Bleeding occurs upon completion of the procedure and removal of the guide sheath. Bleeding may result in hematoma or, in severe cases, to malfunction of critical organs and even death. Such bleeding is generally stopped by the application of digital pressure by a health care professional who applies pressure for a sufficiently long period of time until hemostasis takes place to spontaneously seal the puncture and stop the bleeding. Hemostasis may be augmented by the use of a pressure applying dressing, adhesive-elastic bandages and/or sandbags.

Following inducement of hemostasis hereinafter referred to as “post-hemostasis” pressure needs to be continually applied onto the puncture site. When the blood vessel is the femoral artery, pressure may be maintained for as long as 2-6 hours. At times, recurrent bleeding, particularly if the patient is not in rest, or other complications, such as hematoma and formation of a pseudoaneurysm, may occur. Due to the placement of bandages or the like directly over a puncture site in order to maintain pressure thereat, such complications are liable not to be noticeable at the puncture site, and irreversible bodily damage may result.

It is an object of the present invention to provide a post-hemostasis pressure maintaining device which is configured such that a puncture site is visible, in order to prevent the occurrence of complications.

It is an object of the present invention to provide a post-hemostasis pressure maintaining device by which a puncture site may be monitored.

It is an additional object of the present invention to provide a device by which the total time for applying pre-hemostasis and post-hemostasis pressure onto a blood vessel puncture site may be reduced, relative to the prior art.

Other objects and advantages of the invention will become apparent as the description proceeds.

### SUMMARY OF THE INVENTION

The present invention provides a device for maintaining pressure onto a blood vessel, which comprises a tissue-confining device having two parallel longitudinally extending bars which extend between a proximal end and a distal end, at least one strap attachable to tissue in the vicinity of the blood vessel, for retaining said tissue-confining device in compressing contact with tissue in the vicinity of a blood vessel, and means for affixing said at least one strap to an element connected to said tissue-confining device.

The term “tissue-confining device,” as referred to herein, denotes a device with an open area bounded by its frame, which is externally placed over, or in contact with, a limb of a patient and above a sealed puncture site of the blood vessel, and is so configured that following the application of an axial force to said tissue-confining device it entraps, within said open area, and compresses, tissue in the vicinity of the punctured blood vessel. A tissue-confining device generally comprises two parallel, longitudinally extending bars, interconnected at or adjacent their respective proximal and/or distal ends by at least one arcuate connecting member. The depth to which the tissue in the vicinity of the sealed puncture site is compressed depends on the magnitude of said axial force and the rigidity of said tissue. The tissue-confining device is therefore fixated with respect to said blood vessel.

The term “strap,” as referred to herein, denotes an element which is engageable under tension with tissue external to the tissue-confining device and is preferably, but not necessarily, rectangular. A strap may be an elastic bandage, an adhesive bandage, a belt, cloth, or a combination thereof.

In one embodiment of the invention, the device is a post-hemostasis pressure maintaining device and the tissue-confining device is positioned in the vicinity of a sealed puncture site, the at least one strap being adapted for retaining said tissue-confining device in sufficient compressing contact with tissue in the vicinity of a blood vessel so as to maintain sealing of said puncture site.

Prior to introduction of a guide sheath into a blood vessel, tissue is punctured by a needle held at an inclination with respect to a horizontal plane ranging from 45-60°. After the skin and then the blood vessel are punctured, a wound canal is formed between the skin puncture site and the blood vessel puncture site, which are separated due to the inclination of the tissue-puncturing needle. During hemostasis, the skin puncture site and the blood vessel puncture site are both sealed. Application of axial pressure onto a tissue-confining device reduces the angle of the wound canal, causing surrounding tissue to apply a pressure onto the sealed blood puncture site so as to maintain hemostasis (hereinafter referred to as “post-hemostasis”). Blood flow through the blood vessel is not constricted by the tissue-confining device or by the at least one strap as tissue in the vicinity of the sealed puncture site is compressed.

The tissue-confining device preferably has an open area bounded by spaced apart longitudinally extending bars and at least one connecting bar, the region surrounding the sealed puncture site being visible via said open area.

In one aspect, axial pressure is applied onto the tissue-confining device by means of an artery clamp apparatus. The artery clamp apparatus may be adjust able and said tissue-confining device may be detachable from said adjustable artery clamp apparatus.

The term “artery clamp apparatus,” as referred to herein, denotes an apparatus and a structure that supports said apparatus, which allows for the compressing of tissue in the vicinity of a sealed puncture site of a blood vessel, particularly an artery, by means of a tissue-confining device. An “adjustable artery clamp apparatus” denotes an artery clamp apparatus that may be displaced in a controllable fashion, e.g. wherein the structure is axially and transversally displaceable relative to the sealed puncture site.

As referred to herein, “axial” means a direction from the artery clamp apparatus to a blood vessel, “longitudinal” means a direction parallel to the axis of a brood vessel and “transversal” means a direction perpendicular to the longitudinal direction. “Proximal” means towards the upstream side of blood flow and “distal” means towards the downstream side of blood flow, relative to a puncture site.

In one aspect, manual axial pressure is transmitted to the tissue-confining device by means of an element connected directly or indirectly to the tissue-confining device.

In one aspect, the affixing means comprises proximal and distal strap assemblies which are attached to the proximal and distal ends, respectively, of the tissue-confining device.

Each strap assembly preferably comprises means for angularly displacing and/or for axially displacing at least one strap.

In one embodiment of the invention, the angularly displacing means comprises an axially extending member in fixed relationship with the tissue-confining device by means of a connecting element detachably connected to the tissue-confining device; and an element affixed to said at least one strap which is rotatably attached to said axially extending member and supported by an abutment plate provided with the axially extending member. The axially extending member is preferably a bolt which is threadedly engageable with an internally threaded bolt support connected to, or integral with, the connecting element.

In one aspect, the pressure by which the at least one strap adheres to tissue is adjustable upon lowering or raising the abutment plate by means of the bolt, with the at least one strap remaining at substantially the same angle with respect to the bolt support.

In another aspect, the axially displaceable member is axially displaceable by hydraulic or pneumatic means.

In another embodiment of the invention, each strap assembly comprises a U-shaped element having two mutually parallel, longitudinally extending legs, a connector attached to one of said legs, and an L-shaped element connected at one end to said connector and detachably connected at the other end thereof to a longitudinally extending bar of the tissue-confining device. A strap is securable to a corresponding leg of the U-shaped element and is wrapped around the limb in which the sealed puncture site is disposed.

In another embodiment of the invention, the affixing means comprises at least one track element connected to a rod extending from the tissue-confining device, a strap being translatable and angularly displaceable upon engagement with a corresponding track element. The affixing means may further comprise a U-shaped bracket for receiving a strap through a groove formed in a base thereof, each leg of said bracket being connected to a corresponding track element at a selected transversal distance from a bar of the tissue-confining device.

In one aspect, each strap is insertable into a corresponding groove and foldable, whereby to define two strap portions, the inner face, i.e. the face closer to bodily tissue, of each strap portion being provided with adhesive material.

In another embodiment of the invention, the affixing means comprises a bracket connected to the tissue-confining device, at least one strap assembly being attached to, and being angularly displaceable with respect to, said bracket. A strap assembly may be attached to the bracket by a pin joint or releasably attached to the bracket by a pin vertically protruding from the bracket.

In one aspect, the bracket is U-shaped. A first strap is engaged with a base of the bracket which is transversally spaced from the tissue-confining device and second and third straps are engaged with legs of the bracket which are positioned closer to the tissue-confining device than said base.

In another embodiment of the invention, the affixing means comprises a plate positioned vertically above the tissue-confining device, for receiving two opposed straps in corresponding grooves formed within the plate. Two posts axially extend from the plate to the proximal and distal ends, respectively, of the tissue-confining device.

In an additional embodiment of the invention the device further comprises means for applying a tensile force to the at least one strap.

In one aspect, the tensile force applying means comprises a planar balloon-supporting frame positioned above the tissue-confining device; a plurality of posts connecting said frame and the two longitudinally extending bars of the tissue-confining device; a pad secured to said frame on top of which a balloon is placed; and at least one strap adhered to said balloon and to skin in the vicinity of a sealed puncture site, outward expansion of said balloon being restrained by said at least one strap so as to apply axial pressure to the tissue-confining device.

The balloon is preferably expanded by a syringe in communication with a tube connected to the balloon.

In one aspect, the tensile force applying means comprises a piston assembly. The piston assembly comprises a stationary cylinder having a sealing element on the upper end thereof, said cylinder attached to, or integrally formed with, a lower plate connected to the longitudinal bars of tissue-confining device; a tubular sleeve formed integrally with an upper plate and protruding therethrough, at least one strap being secured to said upper plate; and a unilateral valve seated in a neck formed at the upper end of said sleeve, wherein said sleeve is adapted to be fitted about said cylinder such that the interior volume of said sleeve between said unilateral valve and said sealing element constitutes a pressure chamber, the pressure differential between the interior and exterior of said pressure chamber which is generated following introduction therein of a fluid via said unilateral valve being sufficient to displace said sleeve and upper plate vertically upwardly relative to said cylinder, thereby applying a tensile force to the at least one strap.

In yet another embodiment of the invention, the device further comprises means for applying axial pressure onto the blood vessel.

In one aspect, the means for applying axial pressure onto the blood vessel is an element, such as a relatively small circular element, for applying localized axial pressure (hereinafter referred to as a “plunger”) connected to a rod extending from the tissue-confining device, said plunger being engageable with the blood vessel as the tissue-confining device is brought to compressing contact with the tissue in the vicinity of the blood vessel.

In one aspect, the means for applying axial pressure onto the blood vessel comprises means for applying wide-area axial pressure.

In one aspect, the means for applying extended, wide-area axial pressure onto the blood vessel is an intermediate plate which is fixedly positioned above the tissue-confining device and a syringe-inflatable balloon placeable on the blood vessel and below said intermediate plate, upward expansion of said balloon being restrained by said intermediate plate so as to apply wide-area axial pressure to the blood vessel. The intermediate plate is preferably positioned above the tissue-confining device by means of two posts connecting the two longitudinally extending bars of the tissue-confining device, respectively, and the intermediate plate.

In one aspect, the means for applying extended, wide-area axial pressure onto a sealed puncture site is a lower plate which is wider than the gap between, and connected to, the two bars of the tissue-confining device, wherein subcutaneous tissue confined between the bars of the tissue-confining device projects sufficiently upwardly upon application of manual axial pressure onto the tissue-confining device so as to be pressed by said lower plate and to maintain sealing of the puncture site.

In one aspect, the means for applying extended, wide-area axial pressure onto a sealed puncture site is a pad secured to opposed longitudinally extending bars of the tissue-confining device.

The device preferably further comprises a handle connected to a plate, by which manual axial pressure is transmitted to the tissue-confining device.

In one aspect, the handle is an upper plate larger than, and connected to, the intermediate plate.

In one embodiment of the invention, the device is post-pseudoaneurysm closure maintaining device and the tissue-confining device is positioned in the vicinity of a closed pseudoaneurysm neck, the at least one strap being adapted for retaining said tissue-confining device in sufficient compressing contact with tissue in the vicinity of the closed pseudoaneurysm neck so as to maintain closure thereof.

In one embodiment of the invention, the device is an artery occluding device and the tissue-confining device is positioned in the vicinity of a burst artery wall, total occlusion of the artery being induced by both a proximal plunger positioned adjacent to the proximal end of the tissue-confining device and by axial pressure being applied onto the burst artery wall, the at least one strap being adapted for retaining said tissue-confining device in sufficient compressing contact with tissue in the vicinity of said burst artery wall so as to maintain conglutination of fragmented portions of said burst artery.

The present invention is also directed to a dual hemostasis and post-hemostasis pressure maintaining device, which comprises a tissue-confining device having two parallel longitudinally extending bars which extend between a proximal end and a distal end, means for applying sufficient axial pressure onto said tissue-confining device for inducing hemostasis at a puncture site of a blood vessel, at least one strap attachable to tissue in the vicinity of said puncture site, for retaining said tissue-confining device in sufficient compressing contact with tissue in the vicinity of said puncture site following release of said hemostasis-inducing axial pressure so as to maintain post-hemostasis of said blood vessel, means for affixing said at least one strap to an element connected to said tissue-confining device, and optionally, means for applying a tensile force to the at least one strap.

Preferably, the device further comprises means for applying axial pressure onto the puncture site, such as a plate connected to the two bars of the tissue-confining device and a handle connected to said plate.

In one aspect, subcutaneous tissue confined between the bars of the tissue-confining device projects sufficiently upwardly upon application of the axial pressure onto the tissue-confining device so as to be pressed by the plate and to induce hemostasis.

In one aspect, axial pressure is transmitted to the puncture site by means of an inflatable balloon placeable on the punctured blood vessel and beneath the plate which is connected to the bars of the tissue-confining device, upward expansion of the balloon being restrained by the plate so as to apply wide-area axial pressure to the punctured blood vessel and to facilitate inducement of hemostasis.

In one aspect, the means for applying axial pressure onto the puncture site for inducing hemostasis is also used for maintaining post-hemostasis pressure.

In one aspect, axial pressure is applied onto the tissue-confining device by means of an adjustable artery clamp apparatus, the tissue-confining device being detachable from said adjustable artery clamp apparatus following inducement of hemostasis.

In one aspect, the device further comprises a proximal plunger which is releasably attachable to the proximal end of the tissue-confining device, for applying additional axial pressure to the blood vessel during inducement of hemostasis.

The invention is also directed to a method for maintaining pressure onto a blood vessel, comprising: providing a pressure maintaining device which comprises a tissue-confining device having two parallel bars, at least one strap attachable to tissue in the vicinity of the blood vessel, for retaining said tissue-confining device in compressing contact with tissue in the vicinity of a blood vessel, and means for affixing said at least one strap to an element connected to said tissue-confining device; positioning said tissue-confining device in the vicinity of a blood vessel such that said blood vessel is substantially parallel to, and interposed between, said bars; and applying axial pressure onto said tissue-confining device by said at least one strap.

Axial pressure is preferably applied onto the tissue-confining device by affixing the at least one strap to the element connected to the tissue-confining device and adhering the at least one strap to tissue in the vicinity of the blood vessel.

In one aspect, the pressure maintaining device is a post-hemostasis pressure maintaining device and the tissue-confining device is positioned in the vicinity of a sealed puncture site, whereby the tissue-confining device maintains hemostasis of said sealed puncture site.

In one aspect, the blood vessel is a superficial vein, and particularly, the large saphenous vein. The tissue-confining device is positioned in the vicinity of a corresponding portion of the large saphenous vein, whereby sufficient axial pressure is applied onto said tissue-confining device for ensuring vein closure. A plurality of tissue-confining devices are positioned along the length of the large saphenous vein.

In one aspect, the pressure maintaining device is a post-pseudoaneurysm closure maintaining device and the tissue-confining device is positioned in the vicinity of a closed pseudoaneurysm neck, whereby the tissue-confining device maintains closure of said pseudoaneurysm neck.

In one aspect, the blood vessel is a burst artery and the tissue-confining device is positioned in the vicinity of a burst artery wall, total occlusion of the artery being induced by both a proximal plunger positioned adjacent to the proximal end of the tissue-confining device and by axial pressure being applied onto the burst artery wall, the at least one strap being adapted for retaining said tissue-confining device in sufficient compressing contact with tissue in the vicinity of said burst artery wall so as to maintain conglutination of fragmented portions of said burst artery.

In one aspect, the method further comprises the step of applying a tensile force to the at least one strap.

In one aspect, the tensile force is applied by providing a planar frame positioned above the tissue-confining device and a pad secured to said frame, placing a balloon on top of said pad and adhering a balloon to at least one strap additionally adhered to skin in the vicinity of the blood vessel, and inflating said balloon such that outward expansion of said balloon is restrained by said at least one strap so as to apply axial pressure to the tissue-confining device.

In one aspect, the tensile force is applied by providing a pressure chamber defined by the interior volume of a tubular sleeve between a unilateral valve seated in a neck of said sleeve and a stationary circular sealing element in contact with the inner wall of said sleeve; securing at least one strap to an upper plate integrally formed with said sleeve; attaching said at least one strap to tissue in the vicinity of the blood vessel; and introducing fluid into said pressure chamber via said unilateral valve such that the pressure differential between the interior and exterior of said pressure chamber is sufficient to displace said sleeve and said upper plate vertically upwardly relative to said sealing element, thereby applying a tensile force to said at least one strap.

In another aspect, the method further comprises the step of applying axial pressure onto the blood vessel by an element secured to the tissue-confining device.

The invention is also directed to a method for inducing hemostasis, comprising: providing a hemostasis inducing device which comprises a tissue-confining device having two parallel bars, a plate connected to said two bars, and a handle connected to said plate; positioning said tissue-confining device in the vicinity of a punctured blood vessel such that said blood vessel is substantially parallel to, and interposed between, said bars; applying axial pressure onto said tissue-confining device by said handle, whereby sufficient axial pressure is applied by means of said plate onto said punctured blood vessel so as to induce hemostasis.

In one aspect, subcutaneous tissue confined between the bars of the tissue-confining device projects sufficiently upwardly upon application of the axial pressure onto the tissue-confining device so as to be pressed by the plate and to induce hemostasis.

In one aspect, the method further comprises the steps of placing a syringe-inflatable balloon on the punctured blood vessel and beneath the plate which is connected to the bars of the tissue-confining device; and following application of axial pressure onto the tissue-confining device by the handle, inflating said balloon whereby upward expansion thereof is restrained by said plate so as to apply wide-area axial pressure to the punctured blood vessel and to facilitate inducement of hemostasis.

### BRIEF DESCRIPTION OF THE DRAWINGS

In the drawings:

FIG. 1 is a perspective view of a post-hemostasis pressure maintaining device, according to one embodiment of the invention;

FIG. 2 is a perspective view of the device of FIG. 1 in attachment to an artery clamp apparatus;

FIGS. 3A-C are perspective views of three configurations of a tissue-confining device, respectively;

FIGS. 4 and 5 are perspective views of the device of FIG. 1, illustrating the attachment of a pair of strap assemblies to the tissue-confining device;

FIG. 6 is a perspective view of the device of FIG. 1, illustrating the detachment of the artery clamp apparatus from the tissue-confining device;

FIGS. 7 and 8 are perspective views of another embodiment of a post-hemostasis pressure maintaining device;

FIG. 9 is a perspective view of the device of FIG. 7, illustrating a strap assembly employing more than two angularly displaceable straps;

FIG. 10 is a perspective view of the device of FIG. 7, illustrating the attachment of a pair of strap assemblies to the tissue-confining device;

FIG. 11 is a perspective view of another embodiment of the invention, illustrating track elements by which a strap is translatable and angularly displaceable;

FIG. 12 is a side view of the device of FIG. 11;

FIG. 13 is a perspective view of another embodiment of the invention, illustrating a U-shaped bracket provided with vertically protruding pins by which a corresponding strap assembly is angularly displaceable;

FIGS. 14 and 15 are perspective views of another embodiment of the invention, illustrating a U-shaped and rectangular bracket, respectively, provided with pin joints by which a corresponding strap assembly is angularly displaceable;

FIG. 16 is a perspective view of yet another embodiment of a post-hemostasis pressure maintaining device which is provided with a horizontal plate for receiving two opposed straps;

FIG. 17 is a perspective view from the top of yet another embodiment of a post-hemostasis pressure maintaining device which is provided with a balloon-supporting frame;

FIG. 18 is a perspective view from the bottom of the post-hemostasis pressure maintaining device of FIG. 17, illustrating a pad secured to the balloon-supporting frame;

FIGS. 19 and 20 are top views of the device of FIG. 17, showing a balloon for applying axial pressure;

FIG. 21 is a perspective view from the side of the device of FIG. 17, showing a pad secured to the tissue-confining device for applying extended, wide area pressure onto adjoining tissue;

FIG. 22 is a perspective view of a balloon-assisted dual hemostasis and post-hemostasis pressure maintaining device;

FIG. 23 is a perspective view of another embodiment of a dual hemostasis and post-hemostasis pressure maintaining device;

FIG. 24 illustrates a method in perspective view of attaching a strap to a pressure maintaining device;

FIG. 25 is a perspective view of another embodiment of a dual hemostasis and post-hemostasis pressure maintaining device;

FIG. 26 is an exploded perspective view of the device of FIG. 25;

FIG. 27 illustrates the device of FIG. 25, showing the disc to which a strap is attached at its lowermost position;

FIG. 28 illustrates the device of FIG. 25, showing the disc to which a strap is attached at a vertically displaced position;

FIG. 29 is a perspective view of the device of FIG. 25 which is provided with a proximal plunger;

FIG. 30 is a plan view of an exemplary strap; and

FIG. 31 is a perspective view of en embodiment of a pressure maintaining device having an angularly displaceable strap assembly.

### DETAILED DESCRIPTION OF PREFERRED EMBODIMENTS

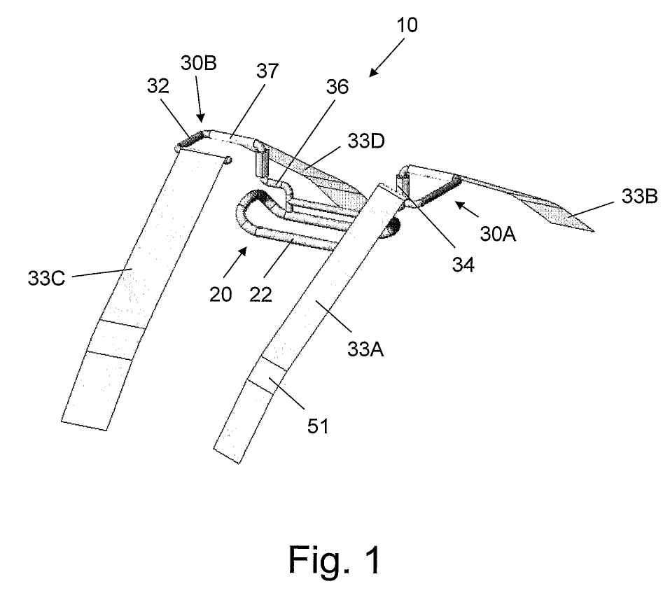

FIG. 1 illustrates a post-hemostasis pressure maintaining device according to one embodiment of the present invention, generally designated as numeral 10, comprising a tissue-confining device generally designated as numeral 20 and a pair of strap assemblies 30A and 30B.

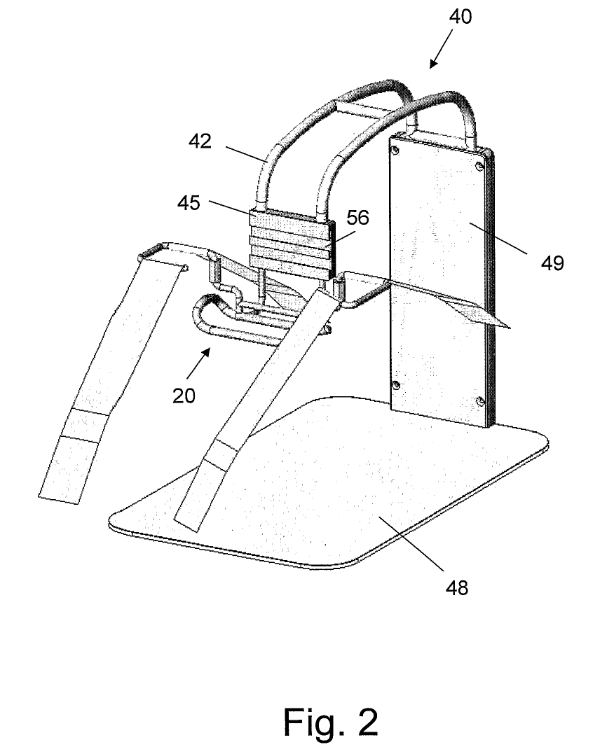

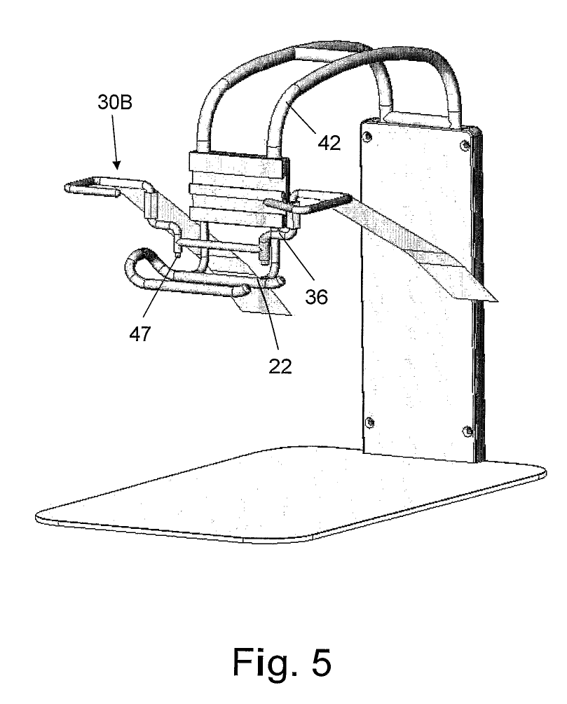

Following the piercing of a blood vessel by an opening of approximately 2-3 mm so that a guide sheath, for example, may be introduced therein during a surgical procedure and the subsequent sealing of the puncture site, tissue-confining device 20 is held adjacent to the sealed puncture site, such that the blood vessel is interposed between, and substantially parallel to, longitudinally extending bars 22. After axial pressure is applied onto tissue-confining device 20 in order to achieve hemostasis, for example by means of artery clamp apparatus 40 (FIG. 2) described in Published International Application WO 03/099350 by the same Applicant, such that the tissue-confining device is fixated with respect to the blood vessel, straps 33A-D are tensed, fixed in position and adhesively attached to the corresponding limb of the patient, so that the tissue-confining device may be retained in compressing contact with tissue in the vicinity of a sealed puncture site. Tissue-confining device 20 is then detached from artery clamp apparatus 40 (FIG. 6), while pressure continues to be applied by the tissue-confining device and the straps to tissue in the vicinity of the sealed puncture site, and particularly to the wound canal formed during the surgical procedure, so that hemostasis may be maintained without constriction of the blood vessel.

At times, the puncture site is at a relatively inaccessible location and the straps cannot be affixed to tissue while being transversally disposed. The present invention provides means for angularly displacing the straps, so that they may be affixed to tissue at a more accessible location.

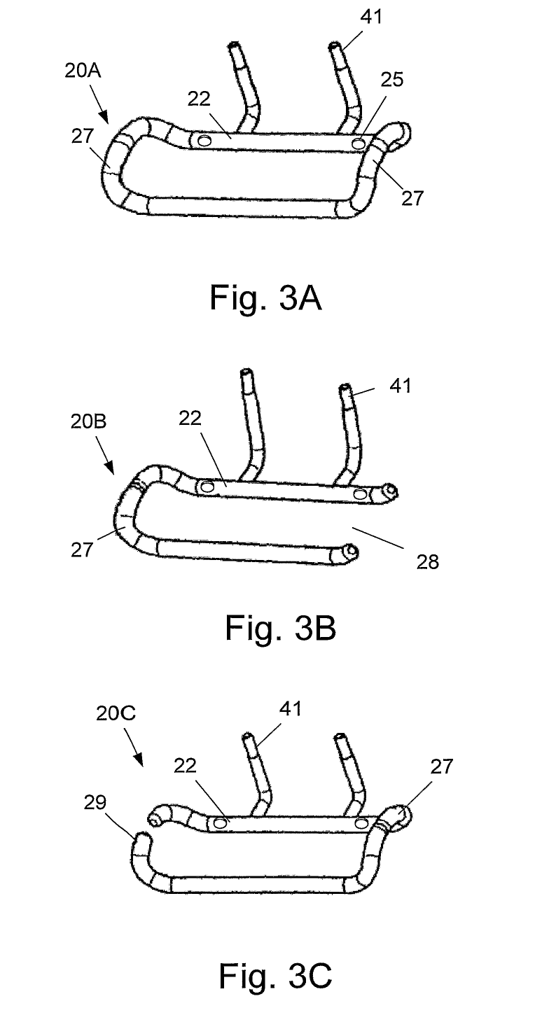

In one embodiment of the invention, each strap assembly 30A and 30B comprises a U-shaped element 32 having two mutually parallel, longitudinally extending legs 37, connector 34 attached to one leg 37 and L-shaped element 36 connected at one end to connector 34 and detachably connected at the other end thereof to tissue-confining device 20 via a suitably shaped aperture 25 formed in bar 22 (FIG. 3A) of the tissue-confining device. Each strap 33A-D is secured to a corresponding leg of U-shaped element 32 by any suitable means well known to those skilled in the art, and is adhesively affixed to tissue outwardly from the tissue-confining device, when the straps are adhesive. When the straps are elastic bandages, each strap may be connected to the opposed strap of the corresponding strap assembly, after being wrapped around the limb in which the sealed puncture site is disposed. As shown, straps 33A and 33B are secured to strap assembly 30A and straps 33C and 33D are secured to strap assembly 30B.

U-shaped element 32 is advantageously pivotable with respect to the corresponding connector 34, so that the straps may be angularly displaced. U-shaped element 32 may also be axially displaceable with respect to tissue-confining device 20 by suitable gripping means (not shown) housed within connector 34.

As shown in FIG. 2, artery clamp apparatus 40 for applying axial pressure onto tissue-confining device 20 in order to achieve hemostasis comprises base plate 48, locking device 49, extendible arcuate arms 42 and adapter 45, which is fixedly connected to the arcuate arms and is detachably connected to tissue-confining device 20 by means of rods 41 (FIGS. 3A-C and 6), e.g. by a pressure fit. Artery clamp apparatus 40, when used, is positioned such that tissue-confining device 20 is disposed directly above a puncture site. As arcuate arms 42 are extended, tissue-confining device 20 is lowered, compressing the tissue of the patient in the vicinity of the sealed puncture site, until the tissue-confining device is fixated with respect to the blood vessel.

Alternatively, axial pressure may be manually applied onto the tissue-confining device, by the hands of a medical professional, if so desired, such that the tissue in the vicinity of the sealed puncture site will be compressed, until the tissue-confining device is fixated with respect to the blood vessel.

Referring now to FIGS. 3A-C, the distance between the two longitudinally extending bars 22 of each tissue-confining device, ranging from 5-80 mm, is selected so that the transversal spacing between a blood vessel, within which a catheter was guided during a recent surgical procedure, and each bar 22 ranges from 0.25-4 cm. With such a transversal spacing, the two bars 22, which are immobilized while being in pressing engagement with tissue and may be supported by a bone in the vicinity of the blood vessel, when the tissue-confining device applies axial pressure to the underlying tissue, the blood vessel is fixated by compressed tissue that is interposed between the blood vessel and each bar 22. Base plate 48 (FIG. 2) placed underneath the limb of the patient further contributes to the stabilization of the tissue-confining device.

In the shown exemplary configurations of a tissue-confining device, each connecting bar 27 which connects the two longitudinally extending bars 22, whether at the distal or proximal end thereof, is provided with a curvature with respect to a vertical plane, such that the connecting bar 27 is elevated above a longitudinally extending bar. This curvature retains mechanical integrity of the tissue-confining device without applying transversal pressure to the blood vessel that would reduce the blood flow therethrough, since the connecting bar is not in contact with the tissue. In FIG. 3A, two connecting bars 27 are employed at the proximal and distal ends, respectively, of tissue-confining device 20A, while in FIG. 3B only one is used, with an opening 28 being formed at the proximal end of tissue-confining device 20B. Opening 28 advantageously allows for the visualization of the puncture site and for the placement and repositioning of an imaging device, whenever necessary. In FIG. 3C, the distal end of tissue-confining device 20C is provided with two ends 29, which are in a spaced, opposed relation with one another, having a curvature with respect to a vertical plane.

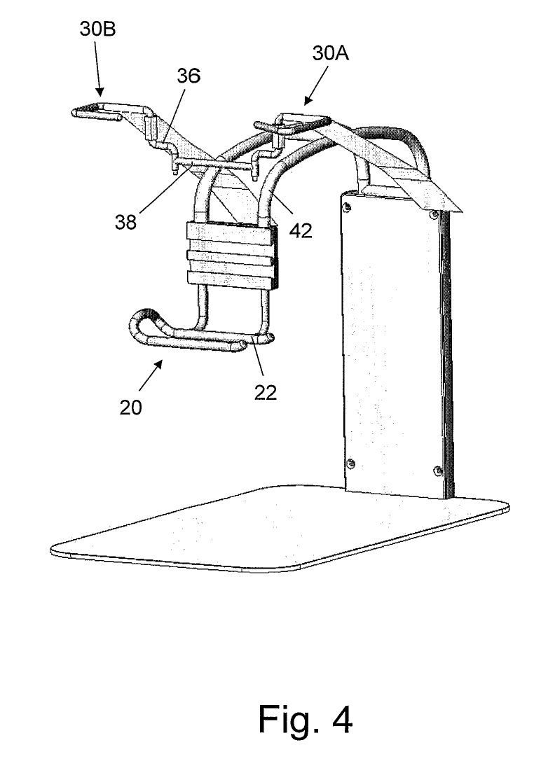

FIGS. 4 and 5 illustrate the attachment of strap assemblies 30A and 30B to tissue-confining device 20. The L-shaped elements 36 of the two strap assemblies 30, respectively, are connected by longitudinally extending cross member 38. Cross member 38 is placed inwardly to arms 42, that is between the arms and the tissue-confining device, as shown in FIG. 4, and then lowered, as shown in FIG. 5, until each pin 47, e.g. a spring-biased pin, formed on the bottom of a corresponding L-shaped element 36 is directly above a corresponding aperture 25 (FIG. 3A) formed in bar 22 of the tissue-confining device. Further lowering of cross member 38 results in the engagement of pins 47 with bar 22, as shown in FIG. 1.

The proximal strap assembly 30A and distal strap assembly 30B are secured to each opposed leg 37 of U-shaped element 32, as shown in FIG. 1. Proximal straps 33A and 33B may be adhesively affixed to tissue, or alternatively, may be wrapped around a different periphery of the limb of the patient, tensed, and are then connected together by attachment means 51, such as Velcro. Distal straps 33C and 33D are connected together in a similar fashion. The straps are engagement with tissue in such a way so as to retain the tissue-confining device in compressing contact therewith. If so desired, one strap may be employed, which is tensed, wrapped around the limb of the patient externally to the tissue-confining device, and affixed to the tissue.

After the straps are properly positioned so as to retain the tissue-confining device in compressing contact with tissue in the vicinity of the sealed puncture site, as described hereinabove, adapter 45 may be detached from tissue-confining device 20, as shown in FIG. 6. Upon retraction of arcuate arms 42, adapter 45 is raised and post-hemostasis pressure maintaining device 10 remains in contact with the limb of the patient. Following detachment of adapter 45 from tissue-confining device 20, the latter continues to be fixated with respect to the punctured blood vessel by means of the tensile force applied by the straps.

Due to the unique configuration of the tissue-confining device, wherein an open area is defined by the area between the longitudinal bars, a physician may clearly view the puncture site via said open area as hemostasis is being maintained. A physician may therefore notice manifestation of a complication during post-hemostasis, such as recurrent bleeding, hematoma and formation of a pseudoaneurysm, therefore preventing irreversible bodily damage.

The open area may also be advantageously utilized for monitoring the sealed puncture site by an imaging device. Since the tissue-confining device is fixated with respect to the blood vessel, an imaging device attached to the tissue-confining device, or being fixedly nested to a seat formed therein may transmit a stable, substantially clear image of the puncture site. The imaging device may be an ultrasound device, such as an X-ray imaging device, for providing required data concerning blood flow through the blood vessel.

With employment of a tissue-confining device described hereinabove such as those illustrated in FIGS. 3A-C, the total time for applying pre-hemostasis and post-hemostasis pressure onto a blood vessel puncture site may be considerably reduced, relative to the prior art.

Published International Application WO 03/099350 by the same Applicant, the content of which is incorporated herein by reference, discloses an apparatus for sealing a puncture in a blood vessel. The apparatus comprises a tissue-confining device, as described hereinabove, which is connected to an adjustable artery clamp apparatus for controllably applying pressure, such as by a fluid circuit, onto the blood vessel. The artery clamp apparatus comprises in a preferred embodiment a proximal plunger and a distal plunger positioned upstream and downstream, respectively, to a skin puncture site. The proximal plunger applies sufficient axial pressure to a blood vessel in order to induce partial or total occlusion, and the distal plunger applies axial pressure directly onto the blood vessel puncture site. During partial occlusion, the diastole and systole temporarily disappear and the blood flow velocity at an arterial puncture site is reduced. Improved coagulation occurs in the absence of vibration or pulsation in the arterial walls, as the blood platelets accumulate easier at the puncture site, thereby reducing the hemostasis time.

Following inducement of hemostasis, the plungers (not shown), which are longitudinally displaceable by means of a corresponding slider 56 (FIG. 2) of rectangular cross section that is slidingly received, by a dovetail arrangement, within a corresponding groove formed within adapter 45 (FIG. 2), may be longitudinally displaced and removed from adapter 45. As the tissue-confining device remains in compressing contact with tissue in the vicinity of the sealed puncture site, the strap assemblies are attached to the tissue-confining device as described hereinabove, straps are wrapped around the corresponding limb of the patient, the artery clamp apparatus is detached from the tissue-confining device, and pressure application to tissue in the vicinity of the sealed puncture site is maintained during post-hemostasis. By employing the same tissue-confining device for both hemostasis and post-hemostasis, valuable time of health professionals is more efficiently utilized.

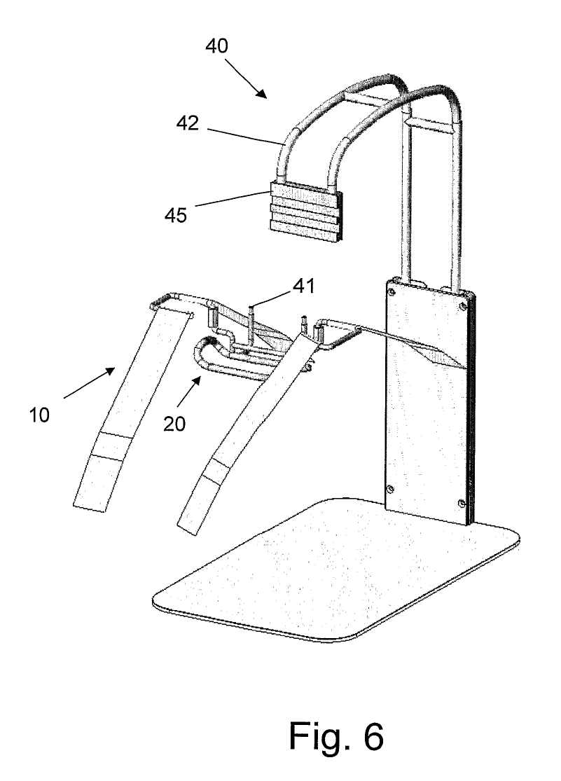

FIGS. 7-10 illustrate another and more preferred embodiment of the invention wherein the strap assembly comprises means for angularly displacing the straps. Each strap assembly 60 comprises connecting element 62 detachably connected to tissue-confining device 20, internally threaded rectangular bolt support 65 integrally formed with, or otherwise fixedly attached to, connecting element 62 in cantilevered fashion, bolt 66 which is threadedly engaged with bolt support 65 and formed with circular abutment plate 69 and head 70, and rectangular strap unifier 72 formed with bore 73. Connecting element 62 has a vertical leg 63 and a horizontal portion 64, and is accordingly arranged so that abutment plate 65 is above and outside of tissue-confining device 20. Straps 75A and 75B, or a single strap if so desired, are affixed to unifier 72 and then adhesively attached to tissue. The straps may be provided with two-sided adhesion, so that one side of the straps may adhere to unifier 72 and the other side of the straps may adhere to tissue in the vicinity of the tissue-confining device. After unifier 72 is lowered on top of abutment plate 69, with head 70 protruding from bore 73, straps 75A and 75B may be angularly displaced with respect to bolt support 65. The pair of straps supported by the abutment plate 69 thereof, are angularly displaceable, so that the straps may be affixed to tissue at a more accessible location. The pressure by which the straps adhere to tissue may be adjusted by lowering or raising bolt 66, while the straps remain at the same angle with respect to bolt support 65.

It will be appreciated that unifier 72 may be rotatably attached to any other suitable axially extending member which is in fixed relationship with tissue-confining device 20. Hydraulic or pneumatic means may be employed to axially displace the axially extending member, in order to adjust the pressure by which the straps adhere to tissue.

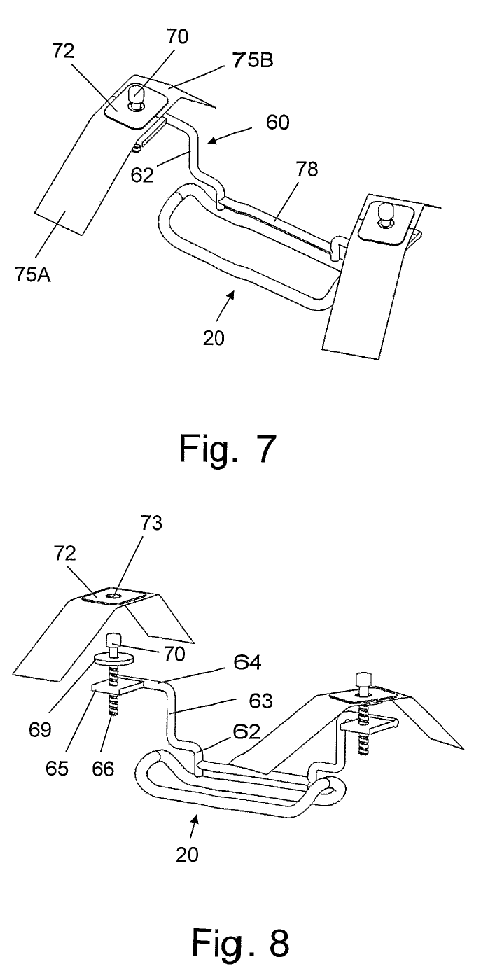

As shown in FIG. 9, each strap assembly 60 may comprise more than one unifier. In the illustrated example, two unifiers are employed, with unifier 77A being affixed to straps 78A and 78B and unifier 77B being affixed to straps 78C and 78D. Straps 77A-C are of sufficient length to be adhesively attachable to tissue outwardly of tissue-confining device 20. Strap 77D has a shortened length, and is used to facilitate affixing to unifier 77B. Unifiers 77A and 77B are both supported by abutment plate 69 (FIG. 8) after being rotatably attached to head 70, and are disposed at different angular dispositions with respect to horizontal portion 64 of the connecting element.

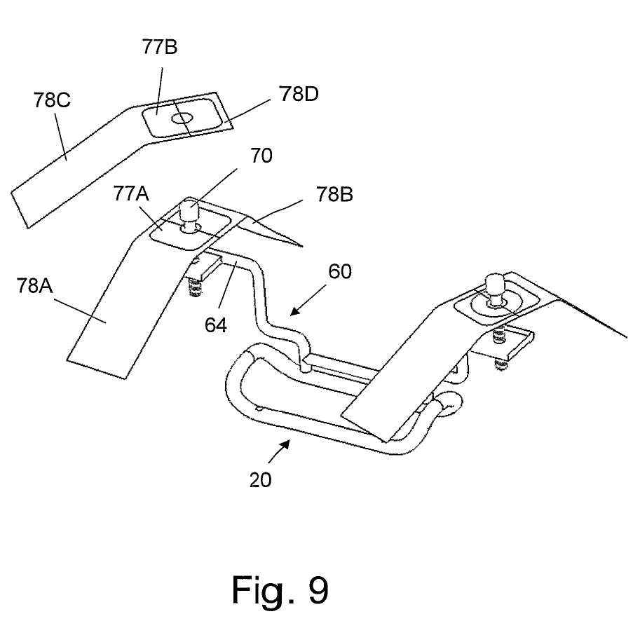

Two connecting elements 62 are connected by longitudinally extending cross member 78. After tissue-confining device 20 is in compressing contact with the tissue in the vicinity of a sealed puncture site of a blood vessel, the two connecting elements 62 and cross member 78 spanning therebetween are lowered, as shown in FIG. 10, until each pin 47 formed on the bottom of a connecting element 62 is directly above a corresponding aperture 25 (FIG. 3A) formed in bar 22 of the tissue-confining device. Further lowering of cross member 78 results in the engagement of pins 47 with bar 22. The connecting elements may be lowered onto the tissue-confining device before straps are placed in supporting relation with each bolt support 65, as shown. Alternatively, each bolt 66 may be threadedly engaged with the corresponding bolt support 65 and unifier 73 may be placed in supporting relation with the corresponding abutment plate 69 before the connecting elements are lowered onto the tissue-confining device.

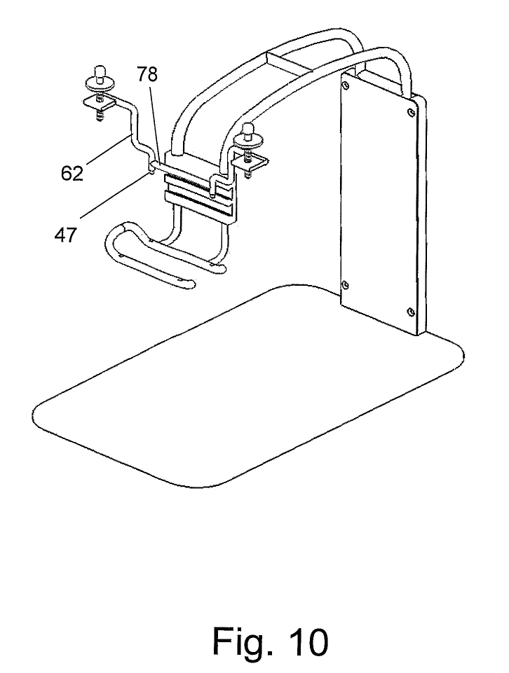

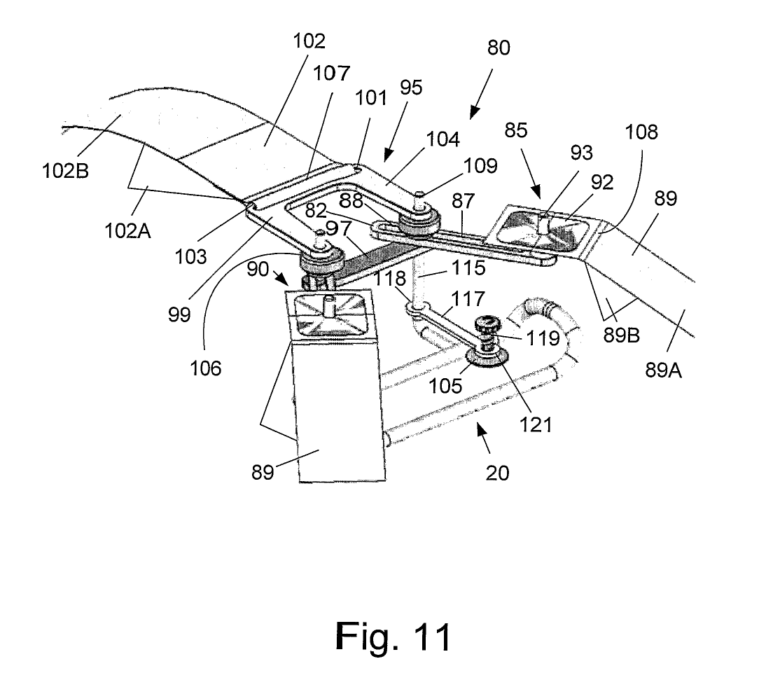

FIGS. 11 and 12 illustrate another embodiment of the invention wherein more than two strap assemblies may be employed, to engage tissue external to the tissue-confining device at multiple and variable locations. Post-hemostasis pressure maintaining device 80 comprises tissue-confining device 20, three strap assemblies 85, 90, and 95, track support 97, and plunger 105.

Strap assemblies 85 and 90 comprise an oval track element 87 having a similarly shaped recess 88, strap 89, and strap unifier 92 in engagement with pin 93 of rotatable pin assembly 94. Pin assembly 94 has an engaging element 96 on its underside, which protrudes through recess 88 and is adapted for engaging pin assembly 94 with track element 87 by a frictional fit. By use of such an arrangement, strap 89 may advantageously be translated to any position along track element 87 and may be angularly displaced relative to the track element.

Strap unifier 92 is formed with a groove, in which strap 89 is insertable. After strap 89 is inserted into groove 108 and folded, two strap portions 89A and 89B are defined thereby. The inner face, i.e. the face that is closer to bodily tissue, of each strap portion is provided with adhesive material. That is, face 89B1 of strap portion 89B and face 89A1 of strap portion 89A are provided with adhesive material, while faces 89A2 and 89B2 are not provided with adhesive material. Accordingly, strap 89 is advantageously affixed to bodily tissue by adhesively affixing face 89B1 of strap portion 89B to one region of a limb of the patient, pulling on strap portion 89A, and adhesively affixing face 89A1 of strap portion 89A, which is in opposed relation to face 89B2, to another region of the limb of the patient.

Strap assembly 95 comprises U-shaped bracket 99, which receives strap 102 through groove 101 formed in base 103 thereof. After a portion of strap 102 is inserted into groove 101 and then folded, loop 107 of the strap is restrained by base 103, thereby forming strap portions 102A and 102B. The inner face of each strap portion is provided with adhesive material. Each leg 104 of bracket 99 is supported by a corresponding bracket connector 106 and is connected thereto by means of upper pin 109 of the bracket connector. Bracket connector 106 allows track element 87 to be maintained at any desired angle relative to track support 97. Each bracket connector 106 has a lower element (not shown) formed with outer threading which protrudes from recess 88 of track element 87. An inner portion of nut 114 is affixed to axially extending rod 115, which is connected to tissue-confining device 20, while an outer portion of nut 114 is engageable with the outer threading of the lower element of the bracket. As nut 114 is tightened, track element 87 is maintained in supporting relation with track support 97, which is integrally formed with rod 115. Accordingly, the spacing between base 103 of the bracket and end 82 of the track element may be adjusted.

Arm 117 connects plunger 105 with rod 115. Since first end 118 of arm 117 is circular, it encircles rod 115 and allows arm 117 to pivot about rod 115. Bolt 119 threadedly engages plunger 105 via second end 121 of arm 117. Arm 117 is pivoted so that plunger 121 may engage the puncture site as tissue-confining device 20 is axially lowered onto the tissue in the vicinity of the punctured blood vessel, thereby affording an additional means for maintaining post-hemostasis pressure.

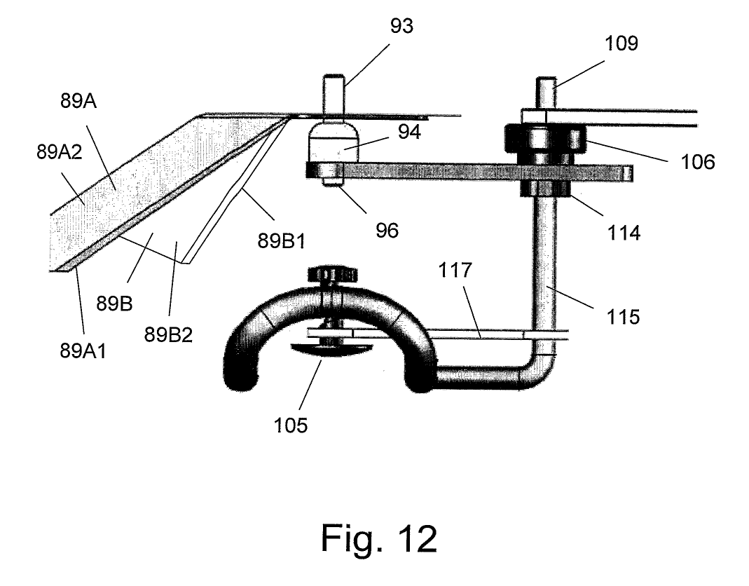

The invention includes other embodiments of a post-hemostasis pressure maintaining device having more than two strap assemblies. In FIG. 13, post-hemostasis pressure maintaining device 130 comprises tissue-confining device 20, U-shaped bracket 135 with which three straps are engageable, and plunger 119. U-shaped bracket 135 has two transversally extending legs 142 and a longitudinal base 138 which is transversally separated from the bars of tissue-confining device 20. Legs 142 of bracket 135 terminate with a pin 144, which is positioned adjacent to, or vertically above, tissue-confining device 20 and which protrudes vertically upwardly from a corresponding leg. Strap unifier 92 of a strap 89 engages a corresponding pin 144, so that strap 89 may be angularly displaced relative to the corresponding leg 142. U-shaped bracket 135 receives strap 102 through groove 137 formed in base 138 thereof after a portion of strap 102 is inserted into groove 137 and folded. Rods 115 connected to tissue-confining device 20 are attached to base 138 of bracket 135 by means of a corresponding bracket connector 139. Arm 117 encircling rod 115 is connected to plunger 119 so that the latter may be angularly displaced about rod 115.

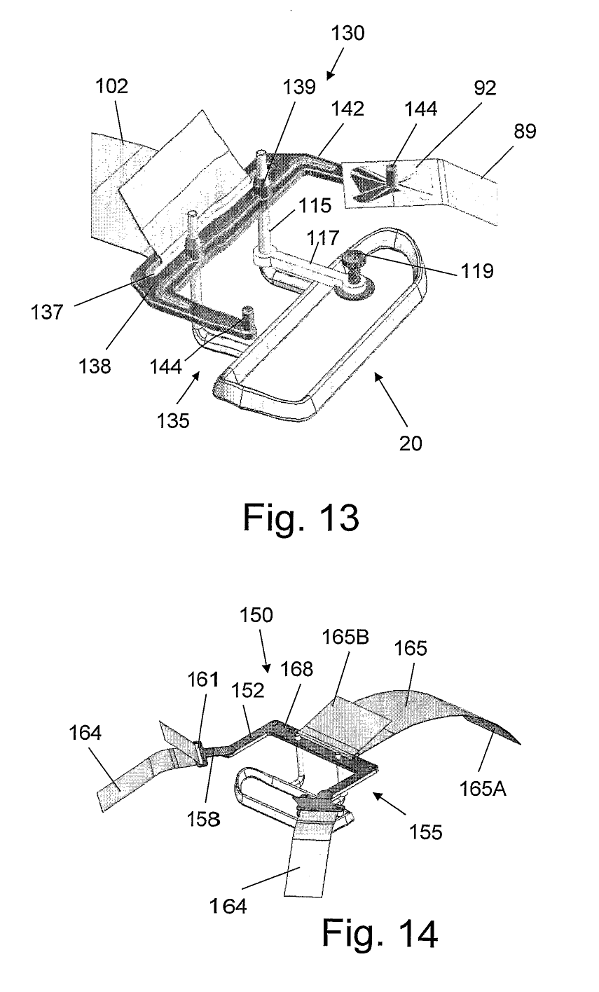

In the embodiment of FIG. 14, post-hemostasis pressure maintaining device 150 comprises U-shaped bracket 155 wherein the two legs 152 thereof terminate with a pin joint (not shown), to which a corresponding strap assembly 158 is pivotally connected. Each illustrated strap assembly 158 has a groove 161 for receiving a corresponding strap 164 therein, but it will be understood that the strap assembly may be of any other configuration which allows a strap to be angularly displaced. A third strap 165 is received in a groove formed in base 168 of bracket 155. Each strap received in a groove has a skin adhering portion and a strap adhering portion. With reference to strap 165, skin adhering portion 165A is adhered to skin in the vicinity of a sealed puncture site. Following adhesion of portion 165A to skin in the vicinity of a sealed puncture site, strap 165 is tensed by pulling on strap adhering portion 165B, whereupon strap adhering portion 165B is adhered to the tensed strap 165.

In the embodiment of FIG. 15, post-hemostasis pressure maintaining device 170 comprises rectangular bracket 175 wherein the two sides 177 thereof terminate with a pin joint (not shown), to which a corresponding strap assembly 158 is pivotally connected.

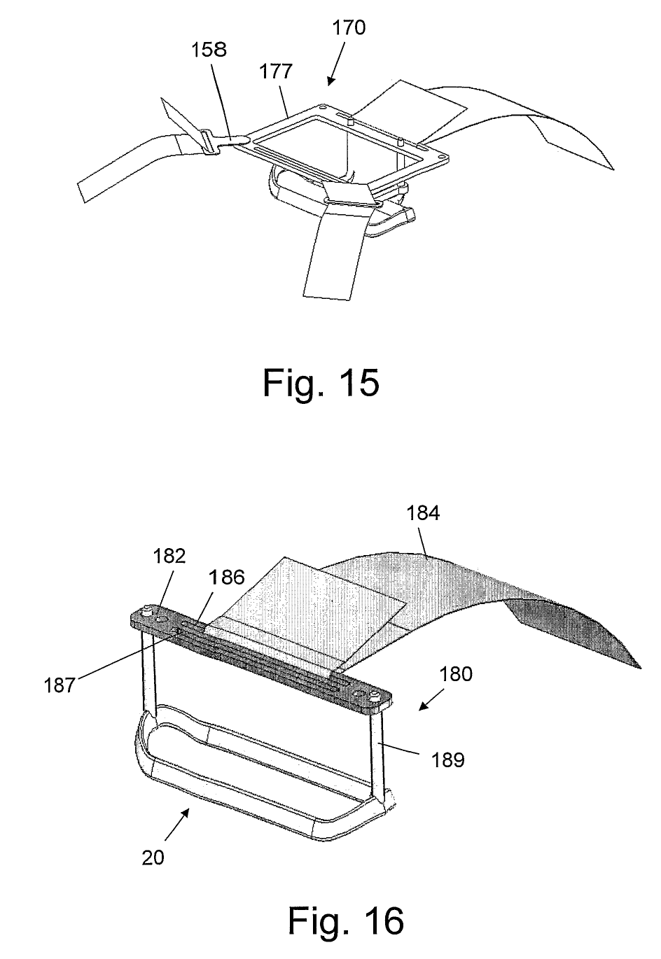

FIG. 16 illustrates another embodiment of the invention wherein post-hemostasis pressure maintaining device 180 comprises a horizontal plate 182 for receiving two opposed straps 184 in grooves 186 and 187, respectively, formed within the plate. Plate 182 is positioned vertically above tissue-confining device 20 by means of two posts 189, which are connected to the proximal and distal ends, respectively, of tissue-confining device 20.

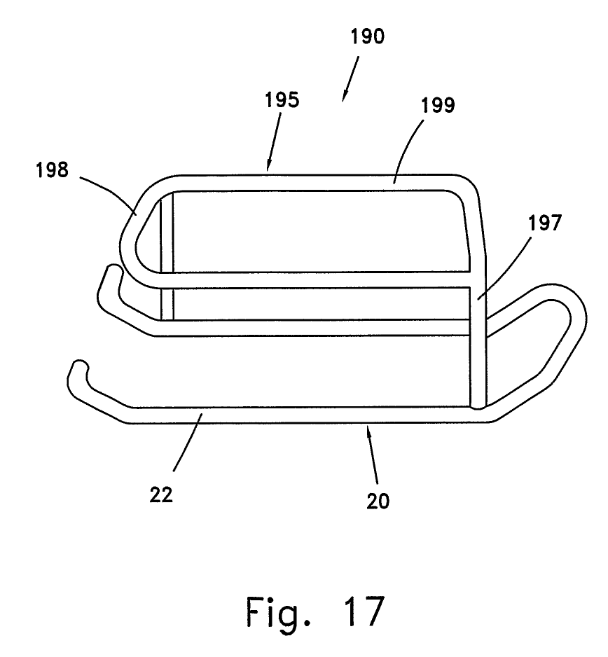

FIGS. 17-21 illustrate yet another embodiment of the invention wherein the tissue-confining device is retained in compressing contact with tissue in the vicinity of a sealed puncture site by means of an inflatable balloon.

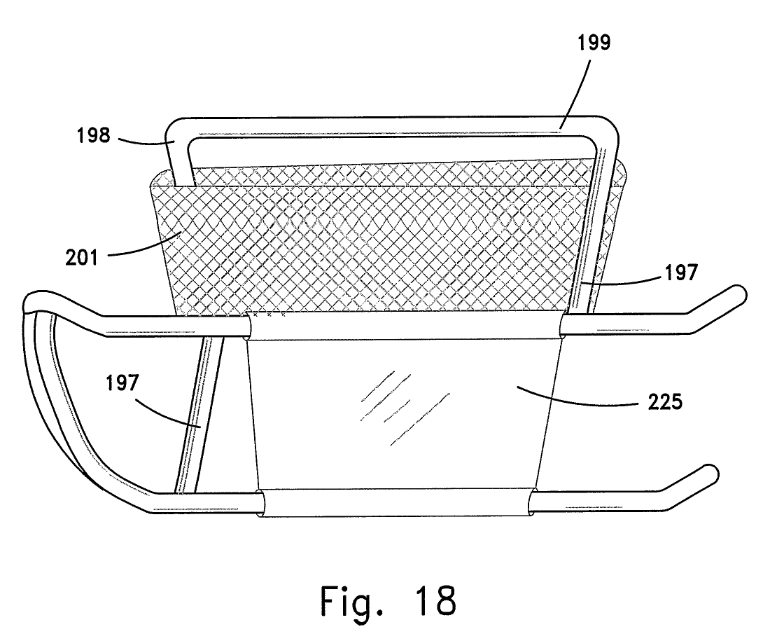

As shown in FIGS. 17 and 18, post-hemostasis pressure maintaining device 190 comprises tissue-confining device 20, planar balloon-supporting frame 195 positioned above the tissue-confining device, and posts 197 connecting frame 195 and the two parallel longitudinally extending bars 22 of the tissue-confining device. Balloon-supporting frame 195 may be of a rectangular configuration as shown having two parallel bars 198 of a relatively shorter length and two parallel bars 199 of a relatively longer length, or may be of any other suitable configuration. A pad 201, e.g. a gauze pad, is secured to two opposed bars 198 of balloon-supporting frame 195. Balloon-supporting frame 195 may be positioned such that bars 199 are longitudinally extending as shown, or may be transversally extending.

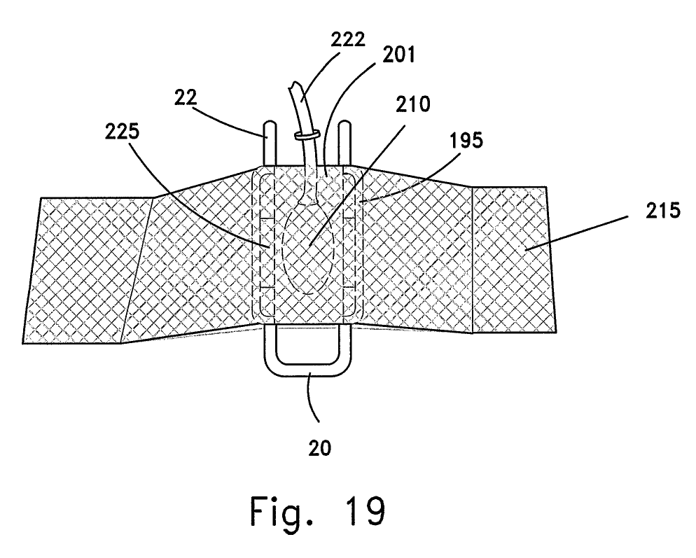

With reference to FIGS. 19-21, balloon 210, which is inflated by means of syringe 220 and tube 222 in communication with both balloon 210 and syringe 220, is placed on pad 201 of balloon-supporting frame 195. While adhesive strap 215 is adhered to skin in the vicinity of a sealed puncture site and to the top of balloon 210, strap 215 applies pressure onto balloon 210. One strap 215 may be employed, as shown in FIG. 19, or two straps, as shown in FIG. 20. While balloon 210 is being inflated, outward expansion thereof is restrained by strap 215, resulting in axial pressure being applied by the balloon to frame 195 and consequently to tissue-confining device 20. The level of axial pressure being applied to the tissue-confining device can be controlled by means of the pressure applied by the syringe. Axial pressure which is continuously applied to tissue in the vicinity of the sealed puncture site and to the wound canal formed during the surgical procedure contributes to the maintaining of hemostasis. Balloon induced axial pressure may be similarly utilized to achieve hemostasis as a puncture site is being sealed.

As shown in FIGS. 18-21, an additional pad 225 may be secured to opposed longitudinally extending bars 22 of tissue-confining device 20. As axial pressure is applied by tissue-confining device 20 to tissue adjoining the sealed puncture site, pad 225 applies extended, wide-area pressure to the sealed puncture site as an additional means for maintaining post-hemostasis pressure.

It will be appreciated that many of the aforementioned post-hemostasis pressure maintaining devices may be modified so as to support a balloon for applying axial pressure to tissue adjoining the sealed puncture site. The axial pressure applied by the balloon may augment the pressure provided by one or more strap assemblies, for maintaining hemostasis. For example, pad 225 may be secured to legs 37 of U-shaped element 32 shown in FIG. 1 or to legs 142 of U-shaped bracket 135 shown in FIG. 13.

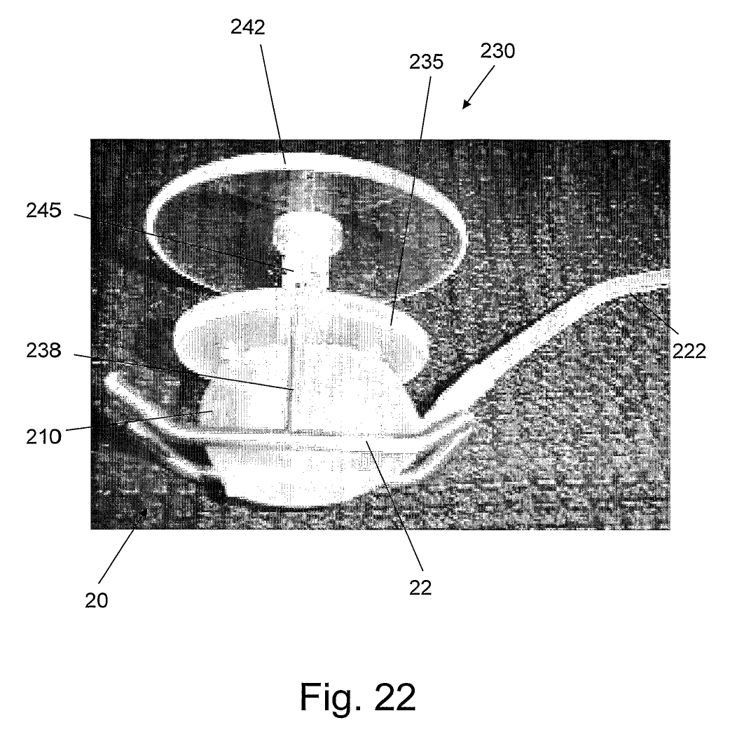

FIG. 22 illustrates another embodiment of a balloon-assisted post-hemostasis pressure maintaining device. Device 230 comprises tissue-confining device 20, intermediate disc 235 positioned above the tissue-confining device, two posts 238 connecting disc 235 and the middle of a corresponding longitudinally extending bar 22 of the tissue-confining device, upper disc 242 having a larger diameter than that of lower disc 235 for applying manual axial pressure, post 245 connecting upper disc 242 and intermediate disc 235, and transparent balloon 210, which is narrower than the gap between the two bars 22 of the tissue-confining device, placed on a sealed puncture site and below intermediate disc 235. It will be appreciated that upper disc 242 and post 245 may be substituted by any suitable handle connected to intermediate disc 235, by which manual axial pressure may be applied onto disc 235.

After axial pressure is applied onto disc 235 and tissue-confining device 20 is fixated with respect to the sealed puncture site, balloon 210 is inflated by means of tube 222 in communication with both balloon 210 and a syringe (not shown). Disc 235 restricts the degree to which balloon 210 rises above the adjoining skin surface, and if the balloon is sufficiently inflated, axial pressure is caused to be applied onto the sealed puncture site by the balloon. One or more adhesive straps applied to upper disc 242 or intermediate disc 235 and to an adjoining skin surface retain tissue-confining device 20 in compressing contact with tissue in the vicinity of the sealed puncture site.

Device 230 may also be advantageously employed for sealing a puncture in a blood vessel. As balloon 210 is inflated to a pressure of approximately 20 mmHg greater than the systolic blood pressure, sufficient axial pressure is applied onto skin puncture site and the blood vessel puncture site so as to achieve hemostasis at the puncture sites. Total or partial occlusion of the blood flow through the blood vessel is maintained for a period of 10-20 minutes as the balloon continues to apply axial pressure, thereby reducing the angle of the wound canal connecting the skin puncture site and the blood vessel puncture site and causing surrounding tissue to apply pressure onto the sealed punctures sites. While a punctured blood vessel which was sealed by prior art methods suffers a risk of complications, such as recurrent bleeding, hematoma and formation of a pseudoaneurysm, due to its lack of visibility to a health professional, the unique configuration of tissue-confining device 20, wherein an open area is defined by the area between the longitudinal bars, allows a health professional to clearly view the puncture sites via said open area. Since balloon 210 is transparent, the puncture sites are not obscured as hemostasis is being maintained, and therefore the health professional may therefore notice manifestation of a complication, therefore preventing irreversible bodily damage. After the health professional determines that hemostasis is achieved, the pressure in balloon 210 is reduced to approximately 50 mmHg and straps are attached to device 230 as described hereinabove, to maintain post-hemostasis pressure of the blood vessel.

The use of tissue-confining device 20 also reduces the time that axial pressure needs to be applied following inducement of total occlusion of the blood flow through the blood vessel: while axial pressure needs to be applied for 4 hours following diagnostic catheterization by prior art methods, axial pressure needs to be applied only for 2-3 hours by the method of the present invention; while axial pressure needs to be applied for 4-8 hours following therapeutic catheterization by prior art methods, axial pressure needs to be applied only for 4-5 hours by the method of the present invention.

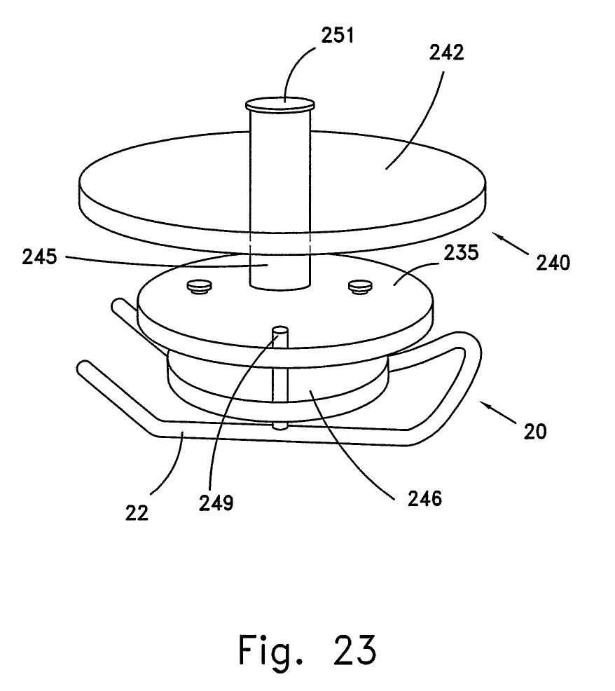

Device 240 illustrated in FIG. 23 may be used to achieve both hemostasis and post-hemostasis. Device 240 comprises tissue-confining device 20, upper disc 242 by which manual axial pressure is applied, intermediate disc 235, post 245 connecting upper disc 242 and intermediate disc 235 and protruding above upper disc 242, and lower disc 246. Lower disc 246, which has a diameter greater than the width between longitudinal bars 22, lies on the two bars 22 and is connected to the latter and to intermediate disc 235 by means of two posts 249. Discs 242, 235, and 246 are made from transparent or translucent material, to allow the health professional to view the open area between the longitudinal bars of the tissue-confining device. As manual axial pressure is applied onto upper disc 242, the pressure is transmitted to tissue-confining device 20 via posts 245 and 249 so as to fixate the tissue-confining device. Tissue below and adjacent to longitudinal bars 22 is compressed, while subcutaneous tissue confined between bars 22 deforms and projects a distance of approximately 1 cm into the open area between the two bars 22. Since lower disc 246 is rigidly attached to longitudinal bars 22, the projected tissue including the skin and blood vessel puncture sites is sufficiently pressed by lower disc 246 so as to induce hemostasis.

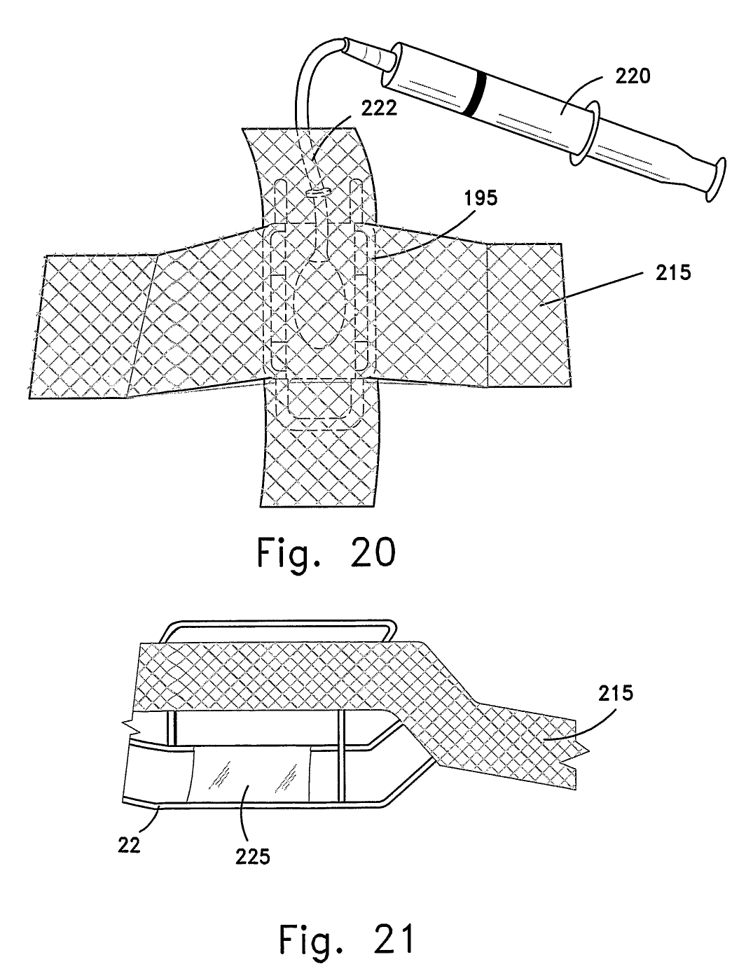

After hemostasis is induced, the manual axial pressure is released and at least one strap is affixed to upper disc 242 and adhered to tissue in the vicinity of the sealed puncture site, to retain the tissue-confining device in compressing contact with tissue in the vicinity of the sealed puncture site and to maintain post-hemostasis. The affixed straps may be in the form of strap assembly 85 shown in FIG. 11 having a strap unifier formed with a bore to engage protruding portion 251 of post 245, in the form of a rectangular strap 215 shown in FIG. 21, which may be adhered to upper disc 242, or in any other suitable configuration.

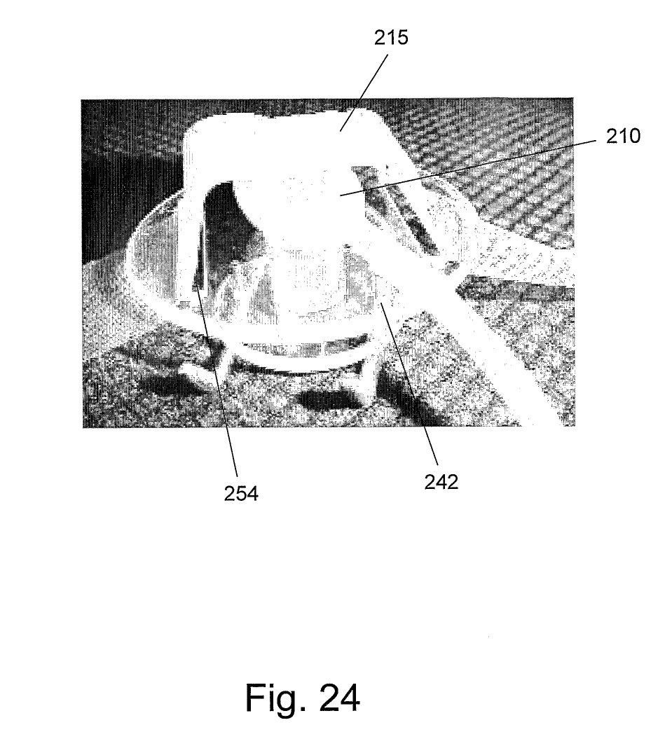

Alternatively, as shown in FIG. 24, post-hemostasis may be maintained by means of a syringe-inflated balloon 210 placed on upper disc 242 and by one or more adhesive straps 215, which are affixed to upper disc 242 and are adhered to the top of balloon 215 and to an adjoining skin surface. Upper disc 242 may be formed with two thin rectangular apertures 254, in each of which a different region of strap 215 is inserted and thereby secured to the upper disc. The portion of strap 215 between the two apertures 254 restrains balloon 210 when being expanded, so as to maintain post-hemostasis pressure as described hereinabove.

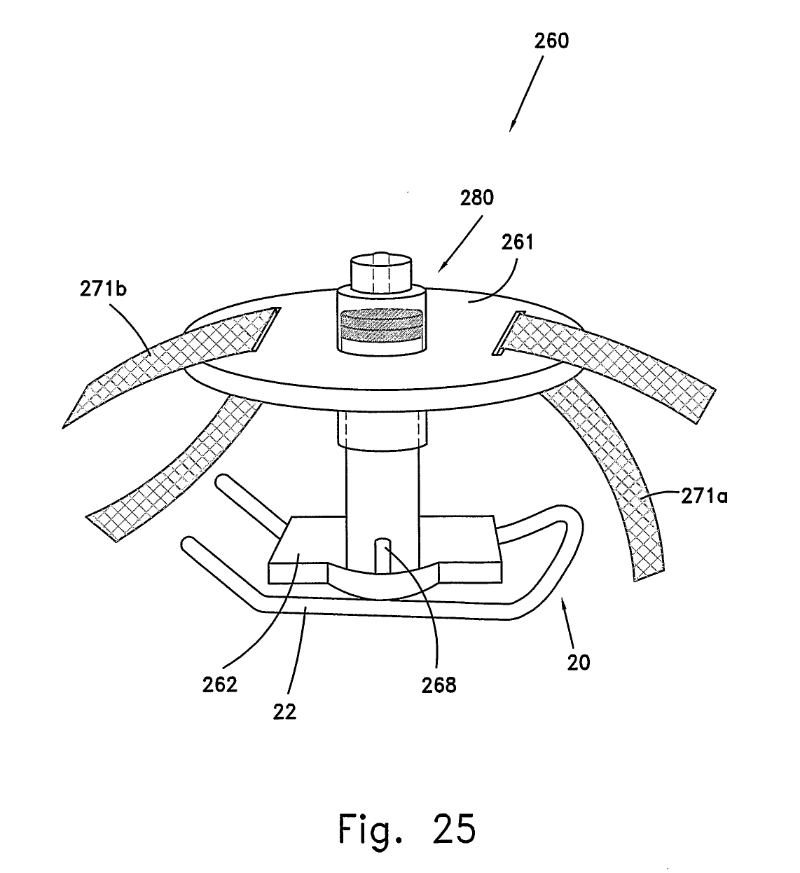

Another embodiment of a syringe-actuated dual hemostasis and post-hemostasis pressure maintaining device is illustrated in FIGS. 25-28.

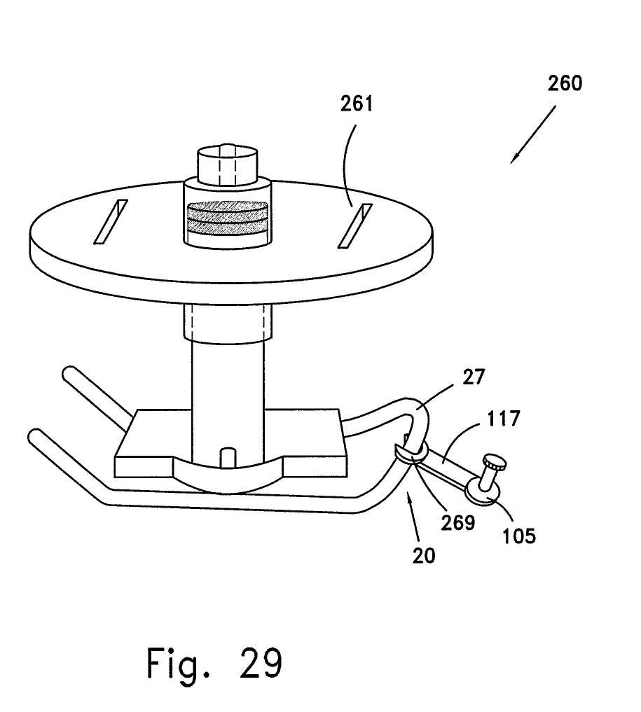

As shown in FIG. 25, device 260 comprises tissue-confining device 20, upper disc 261, straps 271a and 271b affixed to upper disc 261, plate 262 connected to the longitudinal bars of tissue-confining device 20, two posts 268 connecting plate 262 and the middle of a corresponding longitudinally extending bar 22 of the tissue-confining device, and piston assembly 280. Hemostasis is induced by applying manual axial pressure on disc 261, and may be supplemented by means of proximal plunger 105 shown in FIG. 29, which is releasably attachable to connecting bar 27 of tissue-confining device 20 by means of arm 117 and clip 269. Plate 262 may be of any convenient shape, and is shown as being rectangular and having laterally protruding portions on the side thereof to accommodate posts 268. Disc 261 and plate 262 are made from transparent or translucent material, to allow the health professional to view the open area between the longitudinal bars of the tissue-confining device.

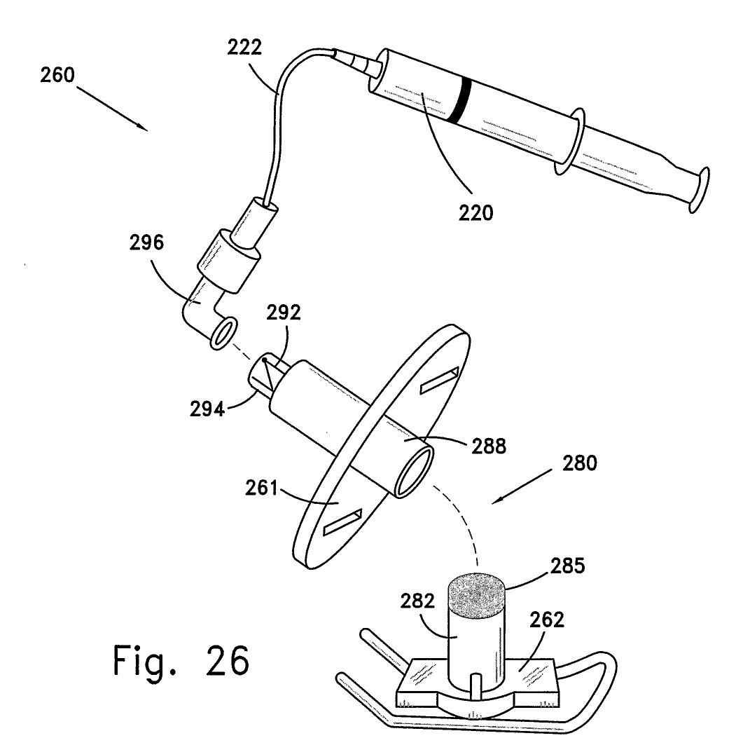

Piston assembly 280 may be seen more clearly in FIG. 26, which is an exploded perspective view of device 260. Piston assembly 280 comprises stationary cylinder 282 attached to plate 262 and having a rubber sealing element 285 on the upper end of the cylinder, tubular sleeve 288 formed integrally with disc 261 and protruding therethrough, and schematically illustrated unilateral valve 292 seated in narrow neck 294 of sleeve 288. Sleeve 288 is adapted to be fitted about cylinder 282 such that the inner diameter of sleeve 288 is slightly less than the diameter of sealing element 285. The interior volume of sleeve 288 between unilateral valve 292 and sealing element 285 constitutes a pressure chamber, which provides the motive force for maintaining post-hemostasis pressure, as will be described hereinafter. Adapter 296 attached to tube 222 of syringe 220 is received in neck 294 of sleeve 288.

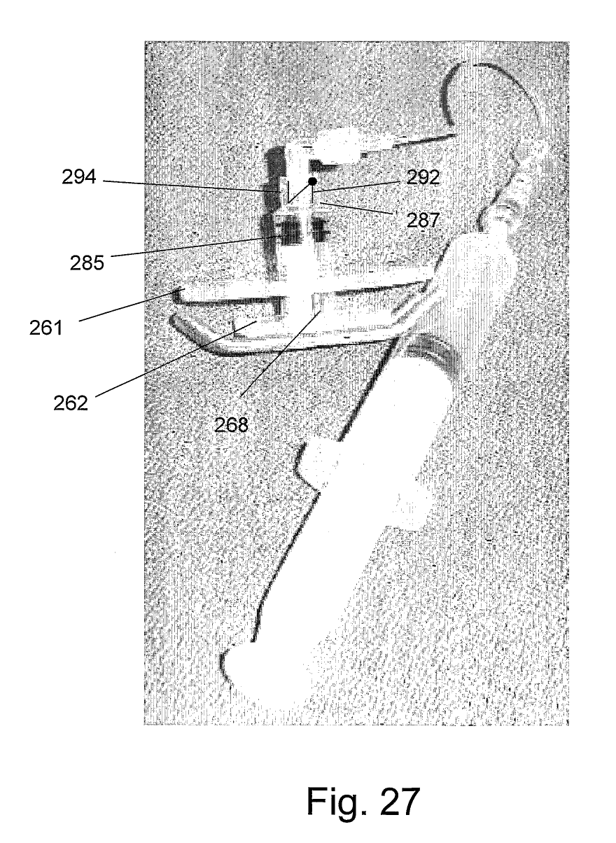

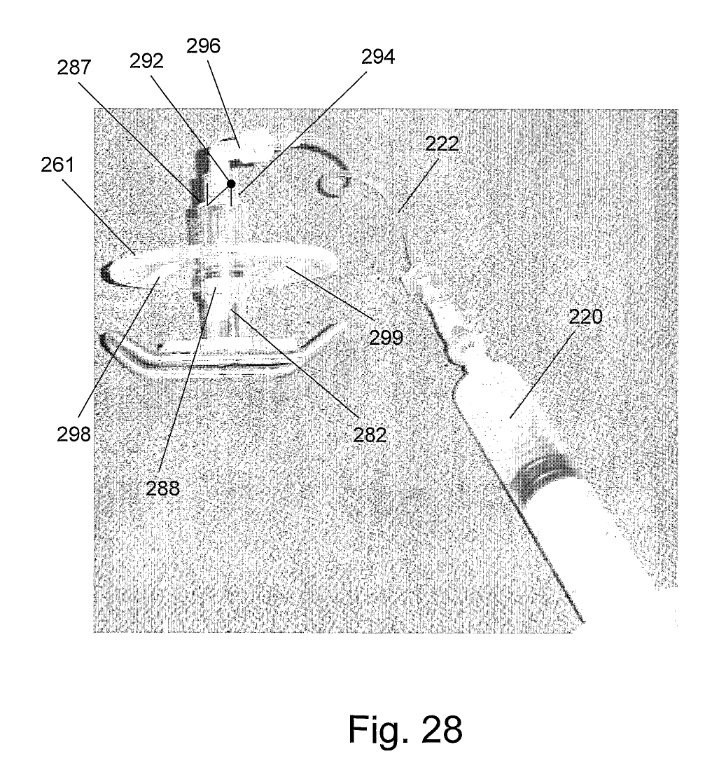

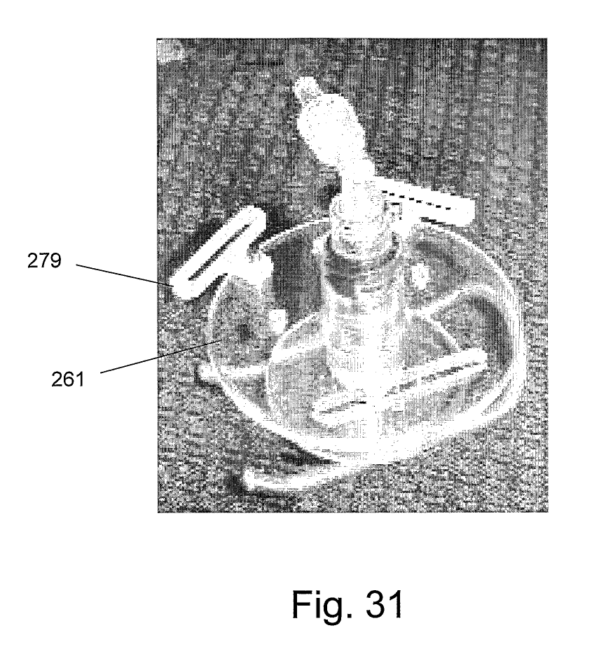

FIGS. 27 and 28 illustrate device 260 prior to, and following, respectively, injection of fluid into the pressure chamber. For clarity, these drawings are shown without attachment of a strap. However, it is understood that a strap may be attached to disc 261 by any suitable means. For example, straps 271a and 271b may be secured to apertures 298 and 299, respectively, as shown in FIG. 25, or a single strap having a strap unifier formed with a bore of a greater diameter than that of sleeve 288 may be inserted into aperture 298, below disc 261, and into aperture 299. More than two straps may be affixed to disc 261, for example, by employing angularly displaceable strap assemblies 279, as shown in FIG. 31.

When the two ends of the strap are adhered to tissue in the vicinity of the sealed puncture site, upward displacement of disc 261 applies a tensile force to the strap.

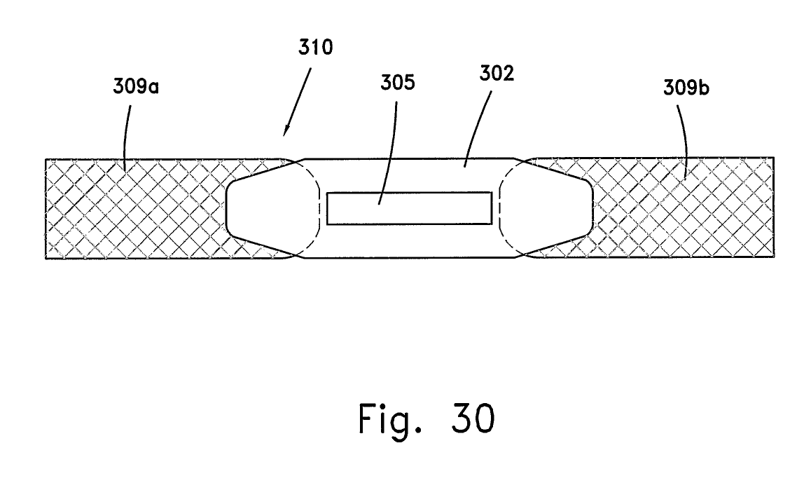

Alternatively, as shown in FIG. 30, a strap 310 having a central portion 302 punched with rectangular aperture 305 and adhesive portions 309a and 309b, which are adhered to the two ends of central portion 302, respectively, may be employed. The width of aperture 305 is greater than the outer diameter of sleeve 288, allowing central portion 302 to be lowered onto the disc while adhesive portions 309a and 309b are adhesively affixed to the disc.

Before fluid is introduced into the pressure chamber, disc 261 is at its lowermost position, as shown in FIG. 27. Posts 268 define the minimum separation between disc 261 and plate 262. In the illustrated example, the length of sleeve 288 between disc 261 and neck 294 is configured such that sealing element 285 contacts the underside of unilateral valve 292 when disc 261 is at its lowermost position. However, other sleeve lengths are also suitable.

As fluid in introduced into the pressure chamber by means of syringe 220 via tube 222, adapter 296, and unilateral valve 292, the fluid within the pressure chamber becomes pressurized, e.g. to a pressure of 20-50 mmHg. Due to the pressure differential between the interior and exterior of the pressure chamber, the pressurized fluid acts on the underside of unilateral valve 292 and of shoulder 287 joining neck 294 to sleeve 288, causing sleeve 288 to be displaced vertically upwardly relative to stationary cylinder 282. As sleeve 288 is displaced vertically upwardly, disc 261 integrally formed therewith is similarly displaced vertically upwardly, thereby applying a tensile force to the strap attached to disc 261. The force applied by the adhesive strap or straps onto tissue is greater than the force applied by the fluid within the pressure chamber.

When the fluid introduced into the pressure chamber is air or hydraulic fluid, a manometer may be installed such that it is in communication with the pressure chamber, in order to monitor and adjust the fluid pressure. Similarly, a manometer may be added to be in communication with balloon-assisted pressurization.

It will be appreciated that unilateral valve 292 need not be seated within neck 294 of sleeve 288, but rather may be positioned e.g. within adapter 296 or tube 222.

The device of the present application is suitable for various applications, in addition to maintaining post-hemostasis pressure. One suitable application of the device of the present invention is hemorrhage control. Following, for example, an automobile accident or a terrorist activity such as a shooting incident, an artery wall of a victim of the accident or terrorist activity bursts at one or more locations to an opening of approximately 3-5 cm. A medic that is dispatched to the accident site needs to quickly achieve temporal occlusion of the burst artery wall, to avoid serious blood loss which could lead to the malfunction of critical organs, and even to death. Medics heretofore have relied on a tourniquet to temporarily stop the flow of blood through a large artery. A significant level of pressure, which is usually produced by twisting a bandage with an elongated element such as a stick, needs to be applied for a long duration onto a burst artery. At times, a medic does not have enough strength to temporarily stop the flow of blood, particularly when the artery is burst at multiple locations. It is therefore of critical importance to a victim to provide a device which can quickly and effortlessly occlude a burst artery.

When a tissue-confining device is positioned proximate to a burst artery such that the artery is substantially parallel to the longitudinally extending bars of the tissue-confining device, application of axial pressure, e.g. by the hands of a medic or by a balloon, onto the tissue-confining device causes adjacent fragmented portions of the burst artery wall to be brought together. Application of axial pressure onto a proximal plunger applies in turn sufficient axial pressure onto the artery so as to induce total occlusion thereof. Temporal occlusion of the artery may be facilitated by a distal plunger or by a pad secured to the longitudinal bars of the tissue-confining device, which presses on the burst portion of the artery wall. Employment of a strap for retaining the tissue-confining device in compressing contact with tissue in the vicinity of the burst artery wall maintains conglutination of the fragmented portions of the burst artery.

The pressure maintaining device of the present invention is also suitable for applying constant pressure to the large saphenous vein, which extends from the foot to the saphenous opening in the broad fascia of the thigh. Following a saphenous vein closure treatment, such as an endovascular laser closure procedure, sclerotherapy, and radio frequency ablation, for valvular incompetence, which is characterized by reflux and which could lead to varicose veins, an elastic bandage is generally applied to the large saphenous vein for 24 hours, to aid the healing process. In lieu of an elastic bandage, a plurality of tissue-confining devices may be deployed along the length of the large saphenous vein, each of which applying axial pressure to adjoining tissue to ensure vein closure. Alternatively, one elongated tissue-confining device having substantially the same length as the large saphenous vein and a transversal spacing of the longitudinal bars thereof sufficient for fixating the vein by interposed compressed tissue may be employed. Similarly, the device of the invention may be employed to apply constant pressure onto any extremity or superficial vein, and particularly, the pressure applied by the device is controllable to ensure proper closure.

The device of the present invention is also suitable for a post-pseudoaneurysm-closure maintaining device. As described in Published International Application WO 03/099350 by the same Applicant, application of an axial force by a proximal plunger proximally to a puncture site and by a distal plunger on the path of blood communication between the artery and the hematoma (commonly referred to as the pseudoaneurysm neck) induces absorption of pseudoaneurysm into an adjacent blood vessel. The proximal and distal plungers (not shown) are longitudinally displaceable by means of a corresponding slider 56 (FIG. 2) of rectangular cross section that is slidingly received, by a dovetail arrangement, within a corresponding groove formed within adapter 45 (FIG. 2). Supplementary axial pressure applied to the pseudoaneurysm neck may be provided by a longitudinally extending bar of the tissue-confining device.

Following closure of the pseudoaneurysm, the plungers may be longitudinally displaced and removed from the adapter. As the tissue-confining device remains in compressing contact with the pseudoaneurysm neck, the strap assemblies are attached to the tissue-confining device as described hereinabove, straps are engageable with the corresponding limb of the patient, the artery clamp apparatus, if used, is detached from the tissue-confining device, and pressure application to the pseudoaneurysm neck is maintained following closure of the pseudoaneurysm. By employing the same tissue-confining device for both pseudoaneurysm treatment and maintaining post-pseudoaneurysm-closure, valuable time of health professionals is more efficiently utilized.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1. A device for maintaining pressure onto a blood vessel, comprising:
a) a tissue-confining device having two parallel longitudinally extending bars which extend between a proximal end and a distal end;
b) at least one strap attachable to tissue in the vicinity of the blood vessel, for retaining said tissue-confining device in compressing contact with tissue in the vicinity of the blood vessel; and
c) means for affixing said at least one strap to an element connected to said tissue-confining device.

2. Device according to claim 1, wherein the tissue-confining device has an open area bounded by spaced apart longitudinally extending bars and at least one connecting bar.

3. Device according to claim 1, which is a post-hemostasis pressure maintaining device such that the tissue-confining device is positioned in the vicinity of a sealed puncture site, the at least one strap being adapted for retaining said tissue-confining device in sufficient compressing contact with tissue in the vicinity of a blood vessel so as to maintain sealing of the puncture site.

4. Device according to claim 1, further comprising means for applying a tensile force to the at least one strap.

5. Device according to claim 4, wherein the tensile force applying means comprises a piston assembly.

6. Device according to claim 5, wherein the piston assembly comprises:
a) a stationary cylinder having a sealing element on the upper end thereof; said cylinder attached to, or integrally formed with, a lower plate connected to the longitudinal bars of tissue-confining device;
b) a tubular sleeve formed integrally with an upper plate and protruding therethrough, at least one strap being secured to said upper plate; and
c) a unilateral valve seated in a neck formed at the upper end of said sleeve, wherein said sleeve is adapted to be fitted about said cylinder such that the interior volume of said sleeve between said unilateral valve and said sealing element constitutes a pressure chamber, the pressure differential between the interior and exterior of said pressure chamber which is generated following introduction therein of a fluid via said unilateral valve being sufficient to displace said sleeve and upper plate vertically upwardly relative to said cylinder, thereby applying a tensile force to the at least one strap.

7. Device according to claim 4, wherein the tensile force applying means comprises:
a) a planar balloon-supporting frame positioned above the tissue-confining device;
b) a plurality of posts connecting said frame and the two longitudinally extending bars of the tissue-confining device;
c) a pad secured to said frame on top of which a balloon is placed; and
d) at least one strap adhered to said balloon and to skin in the vicinity of a sealed puncture site, said at least one strap being tensed upon outward expansion of said balloon, whereby to retain the tissue-confining device in compressing contact with tissue in the vicinity of the blood vessel.

8. Device according to claim 7, wherein the balloon is expanded by a syringe in communication with a tube connected to the balloon.

9. Device according to claim 1, further comprises means for applying axial pressure onto the blood vessel.

10. Device according to claim 9, wherein the means for applying axial pressure onto the blood vessel is plunger connected to a rod extending from the tissue-confining device, said plunger being engageable with the blood vessel as the tissue-confining device is brought to compressing contact with the tissue in the vicinity of the blood vessel.

11. Device according to claim 8, wherein the means for applying axial pressure onto the blood vessel comprises means for applying wide-area axial pressure.

12. Device according to claim 11, wherein the means for applying extended, wide-area axial pressure onto the blood vessel is an intermediate plate which is fixedly positioned above the tissue-confining device and a syringe-inflatable balloon placeable on the blood vessel and below said intermediate plate, upward expansion of said balloon being restrained by said intermediate plate so as to apply wide-area axial pressure to the blood vessel.

13. Device according to claim 12, wherein the intermediate plate is positioned above the tissue-confining device by means of two posts connecting the two longitudinally extending bars of the tissue-confining device, respectively, and the intermediate plate.

14. Device according to claim 11, the means for applying extended, wide-area axial pressure onto a sealed puncture site is a lower plate which is wider than the gap between, and connected to, the two bars of the tissue-confining device, wherein subcutaneous tissue confined between the bars of the tissue-confining device projects sufficiently upwardly upon application of manual axial pressure onto the tissue-confining device so as to be pressed by said lower plate and to maintain sealing of the puncture site.

15. Device according to claim 11, the means for applying extended, wide-area axial pressure onto a sealed puncture site is a pad secured to opposed longitudinally extending bars of the tissue-confining device.

16. Device according to claim 14, further comprising a handle connected to the lower plate, by which manual axial pressure is transmitted to the tissue-confining device.

17. Device according to claim 16, wherein the handle is an upper plate larger than, and connected to the lower plate by which manual axial pressure is transmitted to the tissue-confining device.

18. Device according to claim 1, wherein axial pressure is applied onto the tissue-confining device by means of an artery clamp apparatus.

19. Device according to claim 1, wherein the artery clamp apparatus is adjustable and the tissue-confining device is detachable from said adjustable artery clamp apparatus.

20. Device according to claim 1, wherein manual axial pressure is transmitted to the tissue-confining device by means of an element connected directly or indirectly to the tissue-confining device.

21. Device according to claim 1, wherein the affixing means comprises proximal and distal strap assemblies which are attached to the proximal and distal ends, respectively, of the tissue-confining device.

22. Device according to claim 21, wherein each strap assembly comprises means for angularly displacing at least one strap.

23. Device according to claim 21, wherein each strap assembly comprises means for axially displacing at least one strap.

24. Device according to claim 23, wherein the angularly displacing means comprises an axially extending member in fixed relationship with the tissue-confining device by means of a connecting element detachably connected to the tissue-confining device; and an element affixed to said at least one strap which is rotatably attached to said axially extending member and supported by an abutment plate provided with the axially extending member.

25. Device according to claim 24, wherein the axially extending member is a bolt which is threadedly engageable with an internally threaded bolt support connected to, or integral with, the connecting element.

26. Device according to claim 24, wherein the pressure by which the at least one strap adheres to tissue is adjustable upon lowering or raising the abutment plate by means of the bolt, with the at least one strap remaining at substantially the same angle with respect to the bolt support.

27. Device according to claim 24, wherein the axially displaceable member is axially displaceable by hydraulic or pneumatic means.

28. Device according to claim 21, wherein each strap assembly comprises a U-shaped element having two mutually parallel, longitudinally extending legs; a connector attached to one of said legs; and an L-shaped element connected at one end to said connector and detachably connected at the other end thereof to the longitudinally extending bar.

29. Device according to claim 28, wherein a strap is securable to a corresponding leg of the U-shaped element.

30. Device according to claim 21, wherein a pair of straps is attachable to a corresponding strap assembly, each strap being wrapped around a different periphery of the limb and connected together by an attachment means.

31. Device according to claim 1, wherein the affixing means comprises at least one track element connected to a rod extending from the tissue-confining device, a strap being translatable and angularly displaceable upon engagement with a corresponding track clement.

32. Device according to claim 31, wherein the affixing means further comprises a U-shaped bracket for receiving a strap through a groove formed in a base thereof each leg of said bracket being connected to a corresponding track element at a selected transversal distance from a bar of the tissue-confining device.

33. Device according to claim 1, wherein the affixing means comprises a bracket connected to the tissue-confining device, at least one strap assembly being attached to, and being angularly displaceable with respect to, said bracket.

34. Device according to claim 33, wherein a strap assembly is attached to the bracket by a pin joint.

35. Device according to claim 33, wherein a strap assembly is releasably attached to the bracket by a pin vertically protruding from the bracket.

36. Device according to claim 33, wherein the bracket is U-shaped.

37. Device according to claim 36, wherein a first strap is engaged with a base of the bracket which is transversally spaced from the tissue-confining device and second and third straps are engaged with legs of the bracket, respectively, which are positioned closer to the tissue-confining device than said base.

38. Device according to claim 1, wherein the affixing means comprises a plate positioned vertically above the tissue-confining device, for receiving two opposed straps in corresponding grooves formed within the plate.

39. Device according to claim 38, wherein two posts axially extend from the plate to the proximal and distal ends, respectively, of the tissue-confining device.

40. Device according to claim 1, wherein each strap is insertable into a corresponding groove and foldable, whereby to define two strap portions, the inner face of each strap portion being provided with adhesive material.

41. Device according to claim 1, wherein the affixing means comprises a plate positioned vertically above the tissue-confining device which is formed with two apertures, in each of which a different region of a strap is inserted and thereby secured to said plate.

42. Device according to claim 1, which is a post-pseudoaneurysm closure maintaining device and the tissue-confining device is positioned in the vicinity of a closed pseudoaneurysm neck, the at least one strap being adapted for retaining said tissue-confining device in sufficient compressing contact with tissue in the vicinity of the closed pseudoaneurysm neck so as to maintain closure thereof.

43. Device according to claim 1, which is an artery occluding device and the tissue-confining device is positioned in the vicinity of a burst artery wall, total occlusion of the artery being induced by both a proximal plunger positioned adjacent to the proximal end of the tissue-confining device and by axial pressure being applied onto the burst artery wall, the at least one strap being adapted for retaining said tissue-confining device in sufficient compressing contact with tissue in the vicinity of said burst artery wall so as to maintain conglutination of fragmented portions of said burst artery.

44. A dual hemostasis and post-hemostasis pressure maintaining device, comprising:
a) a tissue-confining device having two parallel longitudinally extending bars which extend between a proximal end and a distal end;
b) means for applying sufficient axial pressure onto said tissue-confining device for inducing hemostasis at a puncture site of a blood vessel;
c) at least one strap attachable to tissue in the vicinity of said puncture site, for retaining said tissue-confining device in sufficient compressing contact with tissue in the vicinity of said puncture site following release of said hemostasis-inducing axial pressure so as to maintain post-hemostasis of said blood vessel;
d) means for affixing said at least one strap to an element connected to said tissue-confining device; and
e) optionally, means for applying a tensile force to the at least one strap.

45. Device according to claim 44, further comprising means for applying axial pressure onto the puncture site.

46. Device according to claim 45, wherein the means for applying axial pressure onto the puncture site is a plate connected to the two bars of the tissue-confining device and a handle connected to said plate.

47. Device according to claim 46, wherein the plate is adapted to sufficiently press subcutaneous tissue confined between the bars of the tissue-confining device and projecting upwardly, upon application of the axial pressure onto the tissue-confining device, so as to induce hemostasis.

48. Device according to claim 46, wherein axial pressure is transmitted to the puncture site by means of an inflatable balloon placeable on the punctured blood vessel and beneath the plate which is connected to the bars of the tissue-confining device, upward expansion of the balloon being restrained by the plate so as to apply wide-area axial pressure to the punctured blood vessel and to facilitate inducement of hemostasis.

49. Device according to claim 45, wherein the means for applying axial pressure onto the puncture site for inducing hemostasis is also used for maintaining post-hemostasis pressure.

50. Device according to claim 45, wherein axial pressure is applied onto the tissue-confining device by means of an adjustable artery clamp apparatus, the tissue-confining device being detachable from said adjustable artery clamp apparatus following inducement of hemostasis.

51. Device according to claim 44, further comprising a proximal plunger which is releasably attachable to the proximal end of the tissue-confining device.

52. A method for maintaining pressure onto a blood vessel, comprising:
a) providing a pressure maintaining device which comprises a tissue-confining device having two parallel bars; at least one strap attachable to tissue in the vicinity of a blood vessel, for retaining said tissue-confining device in compressing contact with tissue in the vicinity of the blood vessel; and means for affixing said at least one strap to an element connected to said tissue-confining device;
b) positioning said tissue-confining device in the vicinity of a blood vessel such that said blood vessel is substantially parallel to, and interposed between, said bars; and
c) applying axial pressure onto said tissue-confining device by said at least one strap.

53. Method according to claim 52, further comprising the step of applying a tensile force to the at least one strap.

54. Method according to claim 53, wherein the tensile force is applied by the steps of:
a) providing a planar frame positioned above the tissue-confining device and a pad secured to said frame;
b) placing a balloon on top of said pad;
c) adhering a balloon to at least one strap additionally adhered to skin in the vicinity of the blood vessel; and
d) inflating said balloon such that outward expansion of said balloon is restrained by said at least one strap so as to apply axial pressure to the tissue-confining device.

55. Method according to claim 52, wherein the tensile force is applied by the steps of:
a) providing a pressure chamber defined by the interior volume of a tubular sleeve between a unilateral valve seated in a neck of said sleeve and a stationary circular sealing element in contact with the inner wall of said sleeve;
b) securing at least one strap to an upper plate integrally formed with said sleeve;
c) attaching said at least one strap to tissue in the vicinity of the blood vessel; and
d) introducing fluid into said pressure chamber via said unilateral valve such that the pressure differential between the interior and exterior of said pressure chamber is sufficient to displace said sleeve and said upper plate vertically upwardly relative to said sealing element, thereby applying a tensile force to said at least one strap.

56. Method according to claim 52, further comprising the step of applying axial pressure onto the blood vessel by an element secured to the tissue-confining device.

57. Method according to claim 52, wherein axial pressure is applied onto the tissue-confining device by affixing the at least one strap to the element connected to the tissue-confining device and adhering the at least one strap to tissue in the vicinity of the blood vessel.

58. Method according to claim 52, wherein the pressure maintaining device is a post-hemostasis pressure maintaining device and the tissue-confining device is positioned in the vicinity of a sealed puncture site, whereby the tissue-confining device maintains hemostasis of said sealed puncture site.

59. Method according to claim 52, wherein the blood vessel is a superficial vein.

60. Method according to claim 59, wherein the blood vessel is the large saphenous vein and the tissue-confining device is positioned in the vicinity of a corresponding portion of the large saphenous vein, whereby sufficient axial pressure is applied onto said tissue-confining device for ensuring vein closure.

61. Method according to claim 60, wherein a plurality of tissue-confining devices are positioned along the length of the large saphenous vein.

62. Method according to claim 52, wherein the blood vessel is a burst artery and the tissue-confining device is positioned in the vicinity of a burst artery wall, whereby the at least one strap retains the tissue-confining device in sufficient compressing contact with tissue in the vicinity of said burst artery wall so as to maintain conglutination of fragmented portions of said burst artery.

63. Method according to claim 62, wherein total occlusion of the artery is induced by both a proximal plunger positioned adjacent to the proximal end of the tissue-confining device and by axial pressure applied onto the burst artery wall.

64. Method according to claim 52, wherein the pressure maintaining device is a post-pseudoaneurysm closure maintaining device and the tissue-confining device is positioned in the vicinity of a closed pseudoaneurysm neck, whereby the tissue-confining device maintains closure of said pseudo aneurysm neck.

65. A method for inducing hemostasis, comprising:
a) providing a hemostasis inducing device which comprises a tissue-confining device having two parallel bars, a plate connected to said two bars, and a handle connected to said plate;
b) positioning said tissue-confining device in the vicinity of a punctured blood vessel such that said blood vessel is substantially parallel to, and interposed between, said bars; and
c) applying axial pressure onto said tissue-confining device by said handle, whereby sufficient axial pressure is applied by means of said plate onto said punctured blood vessel so as to induce hemostasis.

66. Method according to claim 65, wherein subcutaneous tissue confined between the bars of the tissue-confining device projects sufficiently upwardly upon application of the axial pressure onto the tissue-confining device so as to be pressed by the plate and to induce hemostasis.

67. Method according to claim 65, further comprising the steps of:
d) following the positioning of the tissue-confining device, placing a syringe-inflatable balloon on the punctured blood vessel and beneath the plate which is connected to the bars of the tissue-confining device; and
e) following application of axial pressure onto the tissue-confining device by the handle, inflating said balloon whereby upward expansion thereof is restrained by said plate so as to apply wide-area axial pressure to the punctured blood vessel and to facilitate inducement of hemostasis.
